(19) United States
(12) Patent Application Publication
(10) Pub. No.: US 2025/0144373 A1
(43) Pub. Date: May 8, 2025

(54) EQUIPMENT FOR AND METHOD OF FIXING A MEDICAL DEVICE TO A PATIENT

(71) Applicant: Abiomed Europe GmbH, Aachen (DE)

(72) Inventors: Christoph Nix, Aachen (DE); Cristine Rodriguez Brimmers, Aachen (DE); David Schibilsky, Aachen (DE)

(73) Assignee: Abiomed Europe GmbH, Aachen (DE)

(21) Appl. No.: 18/715,561

(22) PCT Filed: Dec. 8, 2022
(86) PCT No.: PCT/EP2022/085066

§ 371 Date: May 31, 2024

(30) Foreign Application Priority Data
Dec. 10, 2021 (EP) 212137590

(51) Int. Cl. A61M 25/06; A61M 25/00; A61M 25/02

(52) U.S. Cl. CPC A61M 25/0662; A61M 25/0021; A61M 25/0043; A61M 25/02; A61M 2025/0233; A61M 2025/024; A61M 2205/04; A61M 2209/088; A61M 2210/04; A61M 2210/12

(57) ABSTRACT

A surgical guide tube (1), a kit comprising a surgical guide tube (1), a system comprising a surgical guide tube (1), a clamping unit comprising a surgical guide tube portion and a method of providing a vascular access to a vessel of a patient comprising the step of providing a surgical guide tube is provided. Therein, the surgical guide tube (1) comprises a body having a proximal end, a distal end and a guide lumen with a longitudinal axis, the guide lumen extending from the proximal end to the distal end of the body and configured for inserting a medical device, in particular a catheter of an intravascular blood pump, through the body (2). The surgical guide tube (1) further comprises a sideways extension (10) defining a planar surface (12a, 12b) which is configured for being attached to a fascia (26) of a patient between a vessel (24) of the patient and skin of the patient. In particular, the planar surface (12a, 12b) of the sideways extension (10) is inclined relative to a longitudinal axis (8) of the guide lumen by an inclination angle of about 30° to 60°, preferably 45°.

The present disclosure relates to a surgical guide tube, a kit, a system, and a clamping unit for as well as a method of fixing a medical device, in particular a catheter of an intravascular blood pump, to a patient.

### BACKGROUND

Intravascular blood pumps for supporting a patient's heart are placed within the heart so as to extend into the left or right ventricle. Intravascular blood pumps comprise a catheter and a pumping device attached to the distal end of the catheter. The pumping device includes a pump section and a drive section for driving an impeller in the pump section. Either electric wiring connects an electric motor of the drive section through the catheter with an external power source outside the patient's body, or a flexible drive cable running through the catheter connects the drive section with an external motor. The pump section includes, in addition to the impeller, a blood flow inlet and a blood flow outlet, wherein the blood flow inlet (or outlet, depending on the flow direction) may be positioned at a distal end of a cannula which may be arranged distally of the impeller. A soft flexible extension attached to the distal end of the pump section may complement the intravascular blood pump.

In particular, an intravascular blood pump which is operated in the left ventricle of the heart is placed across the aortic valve, i.e. the pump section extends from the aorta into the left ventricle. In a surgical procedure, the pump section is introduced into the aorta and left ventricle via the arteria subclavia or arteria axillaris. For this purpose, a vascular graft is attached to the arteria subclavia or arteria axillaris, e.g. by anastomosis, and the pump is then introduced through the graft into the arteria subclavia/axillaris towards the aortic arch and finally across the aortic valve into the left ventricle.

Once the pump section is placed across the heart valve, a plug portion of a repositioning unit, which is slidably mounted on the catheter, is pushed into the graft and then fixed to the graft with its distal end portion and to an outer surface of the patient's skin, e.g. in the shoulder region, with its proximal end portion. For example, the repositioning unit comprises a butterfly fixation having two sideways extensions which can be fixed on the patient's skin by sutures or taping, thereby preventing relative movement between the repositioning unit and the patient's skin. Further, once the repositioning unit is placed in the intended position, the catheter of the pump is fixed inside the repositioning unit so that no relative movement between the repositioning unit and the catheter occurs.

However, while the above-described fixation of the repositioning unit and catheter prevents a relative movement between the repositioning unit and the patient's skin, on the one hand, and between the catheter and the repositioning unit, on the other hand, any movement of the patient's shoulder, i.e. the skin in the shoulder region, may lead to movement of the catheter inside the arteria subclavia/axillaris in which the catheter is placed. This means that, if the patient moves the shoulder, the skin portion which is fixed to the repositioning unit inside which the catheter is fixed may also move, whereby a movement of the catheter inside the vessel and finally a dislocation of the pump in the heart may be caused.

It is an object of the present disclosure to resolve or at least mitigate the disad-vantages of the prior art and, in particular, prevent or at least reduce pump dislocation as a result of movement of the shoulder.

### SUMMARY

According to the present disclosure, a surgical guide tube, a kit, a system, a clamping unit and a method according to the appending independent claims are provided.

According to a first aspect of the present disclosure, a surgical guide tube is provided which has a lumen for insertion of a medical device, e.g. a catheter of an intravascular blood pump, through the surgical guide tube. The surgical guide tube is adapted to be fixed to a patient's deep fascia which is located between the skin and the vessel into which the medical device is to be introduced, e.g. the arteria axillaris and the arteria subclavia. For this purpose, the surgical guide tube comprises a fixation portion at its proximal end, to which the fascia can be fixed by suturing, clamping and the like. The distal end of the surgical guide tube may be attached to a vascular graft which is connected to the target vessel to provide access to the vessel for the medical device to be inserted in the vessel. The vascular graft can be fixed to the vessel e.g. by anastomosis. If the access point to the vessel is in the region of the patient's shoulder, it is beneficial to adapt the geometry of the surgical guide tube to the anatomy of the shoulder. For this purpose, the fixation portion of the surgical guide tube is angled relative to the longitudinal axis of its lumen through which the medical device will be guided such that the fixation portion can be brought into contact with and fixed to the deep fascia between the vessel access and the patient's skin as naturally as possible, i.e. substantially without stretching, twisting or otherwise deforming the natural course of the deep fascia.

Specifically, there is provided a surgical guide tube which is configured for insertion into a vascular graft, wherein the surgical guide tube comprises a body which may have a tubular, particularly linear, shape, the body having a guide lumen with a longitudinal axis, a proximal end and a distal end. The guide lumen extends from the proximal end to the distal end of the body and is configured for inserting a medical device, in particular a catheter of an intravascular blood pump, therethrough. The body of the surgical guide tube further comprises a sideways extension extending sideways from the body and defining a planar surface configured for being attached to the fascia of the patient between a vessel of the patient, preferably a blood vessel and further preferably at least one of the arteria axillaris and the arteria subclavia, and the skin of the patient. The particular fascia preferably surrounds or extends adjacent to the vessel. The planar surface of the sideways extension is inclined relative to the longitudinal axis of the guide lumen by an inclination angle of about 30° to 60°, preferably 40° to 50° and more preferably 45°. Preferably, any surface normal on the planar surface does not intersect the longitudinal axis of the guide lumen.

By said inclination angle, stretching, twisting or otherwise deforming the natural course of the patient's deep fascia during fixation of the deep fascia to the planar surfaces of the sideways extension can be prevented or at least reduced when the surgical guide tube is applied to e.g. the arteria axillaris or the arteria subclavia in the region of the shoulder. Since the surgical guide tube is fixed to the patient's deep fascia, the surgical guide tube can be positioned entirely under the skin. The fascia is a layer of dense connective tissue which surrounds muscles, bones, nerves, and blood vessels of the patient's body. There is minimal relative movement between the deep fascia, which extends very closely to the blood vessel. Thus, fixation of the surgical guide tube to the deep fascia underneath the skin minimizes pump dislocation, because movement of the skin in the patient's shoulder region does not neces-sarily cause movement of the surgical guide tube, i.e. movement of the medical device which is inserted through the surgical guide tube into the vessel can be prevented. Therefore, the risk of dislocation of the intravascular blood pump can be reduced.

Therein, the sideways extension is preferably arranged such that it includes an axis which lies in the planar surface of the sideways extension and extends orthogonally through the longitudinal axis of the guide lumen. In particular, the axis of the planar surface which extends orthogonally through the longitudinal axis of the guide lumen constitutes a virtual rotational axis about which the planar surface was virtually rotated from a position in which the longitudinal axis of the guide lumen would be oriented perpendicular to the planar surface into the actual inclined position in which the longitudinal axis of the guide lumen is oriented at an angle, other than 90°, relative to the planar surface by said inclination angle.

Preferably, the sideways extension is attached to the body in a non-detachable manner and is preferably integrally formed with the body. Thereby, the position of the sideways extension is predetermined, and handling of the surgical guide tube can be simplified.

Alternatively, the surgical guide tube may comprise a separate clamping part, i.e. a clamping part which is not attached to the body in a non-detachable manner, but which is independent of the body of surgical guide tube. The clamping part has a proximal end, a distal end and a clamping lumen extending from the proximal end to the distal end of the clamping part, wherein the sideways extension is attached to the clamping part in a non-detachable manner, preferably integrally formed with the clamping part, and wherein the clamping part is configured for clamping onto or around the body of the surgical guide tube such that the planar surface of the sideways extension is inclined relative to the longitudinal axis of the guide lumen by said inclination angle. Thereby, the position of the sideways extension along the longitudinal axis of the surgical guide tube can be individually determined by the surgeon.

That is, the surgeon can place the clamping part at the desired position along the longitudinal axis of the surgical guide tube.

In a preferred embodiment, the clamping part is configured for clamping onto or around the body of the surgical guide tube such that a longitudinal axis of the clamping lumen of the clamping part is parallel to the longitudinal axis of the guide lumen of the surgical guide tube. This means that the planar surface of the sideways extension is inclined by about 30° to 60° relative to the longitudinal axis of the clamping lumen of the clamping part.

Further preferably, the clamping part is configured for reducing the diameter of the guide lumen of the surgical guide tube when clamped onto or around the body of the surgical guide tube, in particular when the clamping part is placed at a longitudinal position of the surgical guide tube between two bumps, in particular adjacent bumps, or a bump and an adjacent bulge (explained below in more detail) provided on the surgical guide tube. In other words, the clamping part reduces the cross-section of the guide lumen when clamped onto or around the body of the surgical guide tube. Thereby, the medical device can be held in its longitudinal position relative to the guide lumen when the medical device is inserted in the guide lumen. In a preferred embodiment, the clamping part comprises at least two arms extending substantially in opposite circumferential directions and each provided with an interlocking profile and arranged such that a cross-section of the clamping lumen is narrowed when the arms are moved past each other in the respective circumferential directions and interlocked with each other via their interlocking profiles. Specifically, the interlocking profiles may form a ratchet mechanism.

In a preferred embodiment, there are provided two of said sideways extensions which are preferably arranged mirror-symmetrically to each other. The two sideways extensions form two wings which extend in opposite radial directions relative to the longitudinal axis of the guide lumen. Preferably, at a position of the surgical guide tube from where the wings radially extend, a distance between the longitudinal axis of the guide lumen and a radially outermost edge of each of the sideways extensions is between about 5 mm and about 19 mm, preferably between about 8 mm and about 16 mm, and further preferably about 12 mm. This way, a sufficiently large area for attaching the patient's fascia to the planar surfaces of the sideways extensions can be provided while keeping the outermost edge of each of the sideways extensions as close as possible to the longitudinal axis of the guide lumen.

Preferably, a length over which the sideways extension extends in a radial direction (with respect to the longitudinal axis of the guide tube) is at least 3 mm. Particularly preferably, said length is between 3 mm and 10 mm and even more preferably about 5 mm. Further preferably, an extension of the sideways extension in a direction normal to the radial direction is at least 3 mm, particularly preferably between 3 mm and 10 mm and even more preferably about 5 mm. Further preferably, a surface area of the sideways extension is at least 9 mm², particularly preferably between 9 mm₂ and 100 mm² and even more preferably about 25 mm². In case that the sideways extension comprises one or more apertures which open to the surface area of the sideways extension, the opening area of the aperture or apertures is considered to be included in the surface area of the sideways extension. Further preferably, the length over which the sideways extension extends in a radial direction and the extension of the sideways extension in a direction normal to the radial direction are approximately the same. Such a dimensioned sideways extension allows for an easy and safe attachment of the sideways extension to the patient's fascia.

Preferably, the body of the surgical guide tube has an outer diameter between 5 mm and 10 mm, more preferably between 6 mm and 9 mm and even more preferably about 8 mm. The inner diameter of the body, i. e. the diameter of the guide lumen, is preferably between 4 mm and 9 mm, more preferably between 5 mm and 8 mm and even more preferably between 6 mm and 7 mm. This way, the surgical guide tube is particularly suitable for use with a catheter based medical device such as a heart pump.

Preferably, the length over which the sideways extension extends in the radial direction is between 0.6 times and 1 time the outer diameter of the body of the surgical guide tube, more preferably 0.8 times the outer diameter of the body of the surgical guide tube.

These dimensions allow an easy attachment of the sideways extensions to a fascia and at the same time an easy insertion of the surgical guide tube into a vascular graft.

Preferably, the length of the surgical guide tube in its axial direction is between 10 mm and 30 mm, more preferably between 15 mm and 25 mm and even more preferably about 20 mm. This makes the surgical guide tube particularly suitable for being arranged between the vessel, in which the access for the medical device is provided, and the patient's skin.

Preferably, the sideways extension comprises attachment points for the fixation to a fascia such as one or more apertures, recesses or protrusions. For example, the sideways extension comprises an aperture, more preferably two or more of said aperture, which is configured for the insertion of a suture or clamp therethrough so as to facilitate the attachment, e.g. by suturing or clamping, of the surgical guide tube to the fascia of the patient. In particular, a longitudinal axis of the aperture extends in a substantially vertical direction relative to the planar surface of the sideways extension. Therein, the aperture may be substantially D-shaped in a transverse cross-section.

Further preferably, the sideways extension is arranged at a proximal end of the surgical guide tube. In particular, the sideways extension may form the proximal end of the surgical guide tube. Thereby, the total length of the surgical guide tube can be kept short. Alternatively, a proximal bulge surrounds the body and forms a proximal end of the surgical guide tube, wherein the proximal bulge preferably is annular, further preferably has a shape of a circular ring. This is particularly useful when the sideways extension is provided on the separate clamping part.

In a preferred embodiment, the surgical guide tube is provided with one or more circumferential bumps extending radially from its body, each of said bumps surrounding the body and preferably being annular, further preferably having a shape of a circular ring. The bumps may be arranged in spaced-apart relationship along the longitudinal axis of the guide lumen. In other words, neighbouring circumferential bumps are arranged with a distance relative to each other along the longitudinal axis of the guide lumen. The circumferential bumps are useful for providing a seal between the surgical guide tube and a vascular graft, when the surgical guide tube is inserted with its distal end into the vascular graft. The circumferential bumps also provide for predetermined clamping positions for the separate clamping part. For these purposes, it is preferred that there are at least two of said circumferential bumps and that they are aligned in parallel to each other along the longitudinal axis of the guide lumen or have the same outer diameter, or both. Further preferred, some or all of the circumferential bumps are positioned on the surgical guide tube distally of the sideways extension, wherein there are preferably at least two distally positioned circumferential bumps and said at least two distally positioned circumferential bumps are aligned in parallel to each other along the longitudinal axis of the guide lumen or have the same outer diameter, or both.

Preferably, a distal bulge surrounds the body and forms a distal end of the surgical guide tube, the distal bulge preferably being annular, further preferably having the shape of a circular ring. This improves sealing properties and may be useful when the sideways extension is provided on the separate clamping part. Further preferably, all of the circumferential bumps which are positioned proximal of said distal bulge are aligned in parallel to each other along the longitudinal axis of the guide lumen or have the same outer diameter, or both. Also preferably, all of the circumferential bumps which are positioned between the sideways extension and said distal bulge are aligned in parallel to each other along the longitudinal axis of the guide lumen or have the same outer diameter, or both.

Specifically, a circumference of the distal end of the surgical guide tube may lie in a plane which is inclined relative to the longitudinal axis of the guide lumen by about 30° to 60°, preferably 40° to 50° and further preferably 45°. Thereby, the surgical guide tube can be further adapted to the anatomy of the target site, e.g. the patient's shoulder. Further, the plane is preferably inclined, relative to the longitudinal axis of the guide lumen, in the same direction and preferably by the same inclination angle, as the planar surface of the sideways extension. In other words, the plane in which the distal end of the surgical guide tube lies and the planar surface of the sideways extension of the surgical guide tube are preferably in parallel.

In a preferred embodiment, the circumferential bumps on the surgical guide tube are concentrically arranged with the longitudinal axis of its guide lumen, and an outer diameter of each of the bumps relative to the longitudinal axis of the guide lumen is about 0,8 times to about 1,8 times, preferably about 1 time to about 1,6 times, and further preferably about 1,3 times larger than an outer diameter of the body of the surgical guide tube. Specifically, the circumferential bumps may have an outer diameter of about 9 mm and the body of the surgical guide tube may have an outer diameter of about 7 mm.

This means that the bumps have preferably the same outer dimensions. This facilitates the insertion of the surgical guide tube into a vascular graft and provides good sealing and fixing properties for e.g. additional fixing means which are described later. Preferably, also the body of the surgical guide tube has a constant outer diameter relative to the longitudinal axis of the guide lumen.

In particular, all of the circumferential bumps of the surgical guide tube are positioned along the longitudinal axis of its guide lumen at a common mutual distance which is larger than a distance from the proximal end to the distal end of the clamping part. Further preferably, at least one of (a) a distance between the proximal end of the surgical guide tube and one of the circumferential bumps which is located closest to the proximal end of the surgical guide tube and (b) a distance between the distal end of the surgical guide tube and one of the circumferential bumps which is located closest to the distal end of the surgical guide tube is larger than a distance from the proximal end to the distal end of the clamping part. This allows for a high number of predetermined clamping positions for the separate clamping part.

According to a second aspect of the present disclosure, a kit is provided comprising the surgical guide tube as described above and a vascular graft, wherein the vascular graft may be e.g. made of Dacron. The vascular graft has a proximal end, a distal end configured for fixing it to the vessel of the patient, e.g. by anastomosis, and a graft lumen extending from the proximal end to the distal end of the vascular graft, wherein the graft lumen is configured for accommodating a distal section of the surgical guide tube in the proximal end of the vascular graft.

Preferably, the surgical guide tube comprises the one or more circumferential bumps as described above, and an inner diameter of the graft lumen is between 1 time and about 1,2 times the outer diameter of the circumferential bumps, in particular the circumferential bump with the largest outer diameter. Specifically, the graft lumen may have an inner diameter of about 10 mm and the circumferential bumps may have an outer diameter of about 9 mm. This facilitates the insertion of the surgical guide tube into the vascular graft and provides good sealing and fixing properties, e.g. by additional fixing means described later.

In a preferred embodiment, the surgical guide tube comprises the clamping part as described above, and the clamping part is configured for fixing the vascular graft to the surgical guide tube by clamping the clamping part onto the graft while the surgical guide tube is inserted in the graft. Thereby, the graft and the surgical guide tube are fixed to each other and a clearance or gap between the graft and the surgical guide tube is closed so as to seal the graft against the surgical guide tube and avoid blood to flow through said clearance. Therein, the clamping part is preferably configured for reducing the diameter of the guide lumen of the surgical guide tube, i.e. for narrowing a cross-section of the guide lumen by clamping the clamping part onto the vascular graft while the surgical guide tube is inserted in the graft. The clamping occurs preferably at a longitudinal position between two adjacent bumps, between the proximal bulge and the bump which is located closest to the proximal bulge or between the distal bulge and the bump which is located closest to the distal bulge of the surgical guide tube. In this way, after inserting a medical device into the guide lumen, the medical device can be held in a fixed position relative to the guide lumen, i.e. the surgical guide tube. In other words, the medical device is prevented from moving inside the guide lumen.

The kit may further comprise at least one of at least one suture and at least one fixing clamp configured for fixing the vascular graft to an outer circumference of the body of the surgical guide tube when the surgical guide tube is inserted in the graft, in particular at a longitudinal position between two neighbouring bumps, between the proximal bulge and the bump which is located closest to the proximal bulge or between the distal bulge and the bump which is located closest to the distal bulge of the surgical guide tube. Preferably, the suture or fixing clamp is configured for reducing the diameter of the graft, i.e. for narrowing a cross-section of the graft lumen, by applying it to the graft while the surgical guide tube is inserted in the graft, in particular to a reduced diameter which is smaller than the outer diameter of the neighbouring bumps, the proximal bulge and the bump which is located closest to the proximal bulge, or the distal bulge and the bump which is located closest to the distal bulge. Thereby, the surgical guide tube inside the graft is fixed to the graft and a clearance between the outer surface of the surgical guide tube and the inner wall of the graft is reduced or closed.

Preferably, the fixing clamp is configured for reducing the diameter of the guide lumen of the vascular graft, i.e. for narrowing a cross-section of the guide lumen, by applying the fixing clamp to the graft while the surgical guide tube is inserted in the graft, in particular at a longitudinal position between two neighbouring bumps, between the proximal bulge and the bump which is located closest to the proximal bulge, or between the distal bulge and the bump which is located closest to the distal bulge of the surgical guide tube. In this way, after inserting a medical device into the guide lumen of the surgical guide tube, the medical device can be held in a fixed relative position to the guide lumen, i.e. relative to the surgical guide tube, by the fixing clamp. In other words, the medical device is prevented from moving inside the guide lumen of the surgical guide tube.

In a preferred embodiment, the kit may comprise a medical device, in particular a catheter, configured for inserting it into and through the guide lumen of the surgical guide tube, wherein the surgical guide tube is preferably slidable on an outer surface of the medical device. Further, the kit may comprise an intravascular blood pump configured for connecting it to the medical device (catheter) and for inserting it through the vascular graft into the vessel of the patient. If the medical device is a catheter, it is preferable to have the catheter made with a catheter shore hardness of about 55D. The surgical guide tube may be made with a hardness of about 60A. The catheter may be made from e.g. polyurethane or Teflon or silicone. Preferably, the catheter has an outer diameter of about 2 mm to about 5 mm, in particular about 3 mm, which allows a small skin access port and thus prevents infections.

According to a third aspect of the present disclosure, a system configured for fixing a medical device to a patient's body is provided. The system comprises a surgical guide tube configured for insertion into a vascular graft. The surgical guide tube comprises a body, which may have a tubular, particularly linear, shape, the body having a proximal end, a distal end, and a guide lumen with a longitudinal axis, the guide lumen extending from the proximal end to the distal end and configured for inserting a medical device, in particular a catheter of an intravascular blood pump, therethrough. The surgical guide tube further comprises a sideways extension extending sideways from the body and defining a planar surface configured for being attached to a patient's fascia between a vessel of the patient, preferably a blood vessel and further preferably at least one of the arteria axillaris and the arteria subclavia, and the patient's skin. The fascia preferably surrounds or extends adjacent to the vessel. Further, the system comprises a fixator configured for being fixed to the patient's skin and for holding the medical device against the patient's body. The fixator has a central body and two fixator wings extending from the central body in opposite directions, and each fixator wing has a planar surface, the planar surfaces facing in a common direction.

Thereby, a part of said system can be fixed under the skin, namely at the patient's deep fascia between the access vessel and the skin, and another part of the system can be fixed onto an outer surface of the patient's skin to enable a safe fixation of the medical device on the patient's body.

Therein, the surgical guide tube may comprise any one of the features described above with reference to the first and second aspect of the present disclosure. For example, in the surgical guide tube according to the first aspect of the present disclosure, the planar surface of the sideways extension of the surgical guide tube is inclined relative to the longitudinal axis of the guide lumen by an inclination angle of about 30° to 60°, preferably 40° to 50°, and further preferably 45°. This, however, is not a necessary feature of the surgical guide tube in the system according to this third aspect of the present disclosure and can be omitted from the system without departing from the present disclosure.

Further, the system according to this third aspect of the present disclosure may comprise any one of the features of the kit as described above with reference to the second aspect of the present disclosure. For example, the system may comprise the vascular graft, sutures, fixing clamp, medical device, and intravascular blood pump according to the above-described configuration of the kit.

In a preferred embodiment of the system, each of the two fixator wings comprises at least one aperture, preferably two or more apertures, which is configured for insertion of a suture or clamp therethrough so as to attach, e.g. suture or clamp, the fixator wings to an outer surface of the patient's skin. Further preferably, the planar surfaces of the fixator wings are at least partially or completely covered with an adhesive configured for sticking the planar surfaces to the patient's skin. In this way, the attachment of the fixator wings to the patient's skin is facilitated.

Preferably, the system comprises a repositioning unit having a proximal end, a distal end and a repositioning lumen extending from the proximal end to the distal end of the repositioning unit, wherein the repositioning lumen is configured for insertion of the medical device therethrough. The repositioning unit comprises at least one button, or lever, which is accessible from outside the repositioning unit and preferably protrudes to or extends through the circumferential outer surface of the repositioning unit. The button or lever is configured, in a first state, for holding the medical device in its longitudinal position relative to the repositioning lumen and, in a second state, for releasing the medical device upon actuation of the button or lever so as to allow movement of the medical device inside the repositioning lumen. Such a repositioning unit allows for releasable fixation of the medical device, i.e. the surgeon can fixate and release the medical device as needed.

Preferably, the central body of the fixator has a proximal end, a distal end and a fixator lumen extending from the proximal end to the distal end of the central body, wherein the fixator lumen is configured for insertion of the medical device therethrough. Thereby, the medical device can be guided and held inside the fixator lumen with the fixator being attached to the patient's skin.

Further preferred, a tubular extension having an extension lumen is attached to, preferably integrally formed with, the central body of the fixator at a distal end of the central body and configured for inserting the medical device therethrough. Thereby, the medical device can be guided and held inside the extension lumen with the fixator being attached to the patient's skin.

In particular, the repositioning unit may be attached to the proximal end of the central body. Therein, the extension lumen, the fixator lumen and the repositioning lumen may be coaxially aligned and, preferably, have the same diameter, which facilitates the insertion of the medical device through said lumens.

Preferably, the system further comprises the medical device which is insertable through the extension lumen, the fixator lumen and the repositioning lumen, wherein the tubular extension, the fixator and the repositioning unit are slidable on the medical device when the button or lever of the repositioning unit is actuated. Thereby, the repositioning unit can be advanced or retraced to the intended located of fixation together with the tubular extension and the fixator.

In a preferred embodiment, the central body of the fixator forms an arched, i.e. convex, bridge such that the medical device can pass between the central body of the fixator and the patient's skin. In other words, the medical device can pass underneath the bridge with the fixator being applied to the patient's skin. Thereby, the central body of the fixator holds the medical device against the patient's skin, whereas the fixator wings attach the fixator to the patient's skin.

Further preferably, the system may comprise a fixation graft configured for implanting it under the patient's skin and inserting the medical device therethrough. The fixation graft preferably comprises a velour on its outer surface configured for the skin, or the tissue under the skin, to grow into it. In this way, another part of the system, namely the fixation graft, can be fixed under the skin, in addition to the surgical guide tube which is fixed under the patient's skin by means of the sideways extension, thereby providing a further means for fixing, sealing and guiding the medical device on the patient's body.

In a preferred embodiment, a tubular extension having an extension lumen is attached to, preferably integrally formed with, the surgical guide tube at the proximal end of the surgical guide tube and configured for insertion of the medical device therethrough. Thereby, the medical device can be guided and held inside the extension lumen.

Preferably, the system comprises a connecting portion having a connecting lumen configured for insertion of the medical device therethrough, the connecting portion connecting a proximal end of the tubular extension with a distal end of the repositioning unit, wherein the connecting portion has a section with a reduced outer diameter, said section being positioned between the repositioning unit and the tubular extension so as to form a waist portion between the repositioning unit and the tubular extension. The waist portion facilitates attachment of the connecting portion to the patient's skin, thereby also fixing the repositioning unit and the tubular extension to the patient's skin. Specifically, the central body of the fixator may form an arched, i.e. convex, bridge such that only the waist portion passes between the central body and the patient's skin. In other words, the waist portion can pass under the bridge with the fixator being applied to the patient's skin. Therein, the waist portion has a proximal end and a distal end and the waist portion can move under the bridge along a direction, which is the direction of the longitudinal length of the connecting lumen, wherein movement of the waist portion is stopped at the distal end and at the proximal end of the waist portion, respectively. Thereby, the waist portion provides a range of movement for the connecting portion within which the connecting portion can be pushed back and forth while at the same time being attached to the patient's skin.

Preferably, the extension lumen, the connecting lumen and the repositioning lumen are coaxially aligned and, more preferably, have the same diameter, which facilitates the insertion of the medical device through said lumens.

According to a fourth aspect of the present disclosure, a clamping unit is provided and configured for fixing a medical device, in particular a catheter of an intravascular blood pump, to a patient's body. The clamping unit has a tubular body with a proximal end, a distal end and a lumen extending from the proximal end to the distal end. The lumen is configured for insertion of the medical device therethrough, i.e. through the clamping unit. The tubular body comprises a surgical guide tube portion at its distal end configured for insertion into a vascular graft and a sideways extension defining a planar surface configured for being attached to a patient's fascia between a vessel of the patient, preferably a blood vessel and further preferably at least one of the arteria axillaris and the arteria subclavia, and the patient's skin. The fascia preferably surrounds or extends adjacent to the vessel. The sideways extension of the tubular body is provided proximal of the surgical guide tube portion. The tubular body further comprises a handling portion provided proximal of the sideways extension and a rotatable tube configured for insertion of the medical device therethrough. The rotatable tube is provided inside the lumen of the tubular body and operatively connected to a first lever, or first button, which is accessible from outside the handling portion. The first lever or first button preferably protrudes to or extends through an outer surface of the handling portion such that actuation of the first lever or first button causes rotation of the rotatable tube about its longitudinal axis in a non-twisting manner. Further, the tubular body comprises a flexible tubular foil portion configured for insertion of the medical device therethrough, wherein the tubular foil portion is provided inside the lumen of the tubular body and fixed to the rotatable tube at a proximal connecting portion thereof such that the tubular foil portion and the rotatable tube are fixed to each other in a non-rotatable manner at the foil portion's proximal connecting portion. Since, further, the foil portion is fixed to the lumen of the tubular body, more specifically to an inner wall of the tubular body, at a distal connecting portion thereof such that the foil portion and the tubular body are fixed to each other in a non-rotatable manner at the foil portion's distal connecting portion, the foil portion twists about its longitudinal axis when the rotatable tube is caused to rotate in a first direction and untwists when the rotatable tube is caused to rotate in a second direction opposite to the first direction after the rotatable tube has been rotated in the first direction. Upon twisting of the foil portion, the inner diameter of the foil portion decreases and, upon untwisting, the inner diameter increases back to its original size. Thus, the clamping unit enables a releasable fixation of the medical device inserted through the lumen of the tubular body, i.e. the surgeon can fixate and release the medical device as needed.

More specifically, the handling portion may further comprise a second button, or second lever, which is accessible from outside the handling portion, preferably protruding to or extending through the circumferential outer surface of the handling portion. The second button or second lever is configured, in a first state, for holding the medical device in its longitudinal position relative to the lumen of the tubular body of the clamping unit and, in a second state, for releasing the medical device upon actuation of the second button or second lever so as to allow movement, longitudinally and rotationally, of the medical device relative to the lumen of the tubular body of the clamping unit. For instance, it is also possible to provide the handling portion additionally with a torsion spring by which the second button or second lever is biased into the first state. Thereby, the foil portion can be configured to fixate the medical device unless the second button or lever is actively actuated into the second state, e.g. by the surgeon, against the spring force.

Further, the handling portion may comprise along its length a section with a reduced outer diameter so as to form a waist portion, wherein the first button or first lever is preferably provided within the waist portion and, further preferably, does not radially extend beyond portions of the handling portion adjacent to the waist portion. Thereby, unintentional operation of the first button or first lever can be avoided.

Preferably, the clamping unit further comprises a fixator configured for fixation to the patient's skin and holding the clamping unit to or against the patient's body. The fixator has a central body and two fixator wings extending from the central body in opposite directions. Each fixator wing has a planar surface, the surfaces facing in a common direction. The central body of the fixator forms an arched, i.e. convex, bridge such that only the waist portion of the handling portion of the clamping unit's tubular body passes between the central body of the fixator and the patient's skin. In other words, only the waist portion can pass underneath the bridge with the fixator being applied to the patient's skin. Therein, the waist portion has a proximal end and a distal end, and the tubular body of the clamping unit's handling portion can move under the bridge along a longitudinal direction, which corresponds to the longitudinal axis of the lumen of the tubular body, until such movement is stopped at the distal end and at the proximal end of the waist portion, respectively. Thereby, the waist portion provides a range of movement for the clamping unit's handling portion within which the handling portion can be pushed back and forth while at the same time being attached to the patient's skin.

Further preferred, the surgical guide tube portion, the sideways extension and the fixator of the clamping unit can comprise any one of the features of the surgical guide tube, the sideways extension and the fixator of the system described above with reference to the third aspect of the present disclosure, respectively.

According to a fifth aspect of the present disclosure, there is provided a method of providing a vascular access to a vessel of a patient, preferably a blood vessel and further preferably at least one of the arteria axillaris and the arteria subclavia. The method comprises the step of providing a surgical guide tube comprising a tubular, in particular linear, body having a proximal end, a distal end, and a guide lumen with a longitudinal axis, the guide lumen extending from the proximal end to the distal end of the body and configured for insertion of a medical device, in particular a catheter of an intravascular blood pump, therethrough, and the further step of fixing the surgical guide tube to a fascia of the patient between the vessel and the skin of the patient, wherein the fascia preferably surrounds or extends adjacent to the blood vessel.

With said method it is possible to fixate the surgical guide tube under the patient's skin such that movement of the patient's shoulder, which might induce movement of the skin in the shoulder region, brings a lower risk of dislocating the surgical guide tube.

Preferably, the surgical guide tube further comprises a sideways extension extending sideways from the body and defining a planar surface, and the method further comprises the step of attaching the planar surface of the sideways extension to the patient's fascia, preferably by suturing, clamping or sticking the sideways extension to the fascia. This provides a simple attachment option. Therein, the planar surface which is attached to the fascia may be a proximal or front planar surface of the sideways extension. Alternatively or additionally, the planar surface which is attached to the fascia may be a distal or back planar surface of the sideways extension.

Further preferably, the surgical guide tube may comprise any one of the features of the surgical guide tube of the system described above with reference to the third aspect of the present disclosure.

In a preferred embodiment, the method further comprises the step of fixing a distal end of a vascular graft to the patient's vessel, e.g. by anastomosis, in particular prior to fixing the surgical guide tube to the fascia. The vascular graft may be made of Teflon or Dacron. It may have a graft lumen extending from the proximal end to the distal end of the vascular graft, wherein the graft lumen is configured for accommodating a distal section of the surgical guide tube in a proximal end of the vascular graft. Thereby, the vascular graft provides access to the vessel into which the medical device is supposed to be inserted.

In particular, the method may further comprise the step of introducing an intravascular blood pump comprising a catheter at its proximal end into and through the vascular graft, preferably after the vascular graft has been fixed to the vessel, and further into the patient's vessel. Preferably, the method further comprises the step of advancing the intravascular pump to the intended final position, which may be the left ventricle of the patient's heart. Further preferably, the surgical guide tube may be slidably mounted on an outer surface of the catheter, and the method may further comprise the step of sliding the surgical guide tube on the outer surface of the catheter such that a distal section of the surgical guide tube enters into the graft lumen of the vascular graft, wherein this step is preferably carried out prior to fixing the surgical guide tube to the fascia. Therein, an inner diameter of the graft lumen may be between 1 time and about 1,2 times, preferably about 1,1 times, an outer diameter of the surgical guide tube, which facilitates the insertion of the surgical guide tube into the vascular graft and sealing of the vascular graft against the surgical guide tube, as will be described later.

Preferably, the method may further comprise the step of fixing the vascular graft to an outer surface of the surgical guide tube, after the surgical guide tube has been inserted into the graft lumen of the vascular graft. The fixation is preferably achieved by clamping or suturing the vascular graft to the outer surface of the surgical guide tube, preferably prior to fixing the surgical guide tube to the fascia. Thereby, the vascular graft can be fixed to and at the same time sealed against the surgical guide tube.

For this purpose, the surgical guide tube may further comprise a separate clamping part having a proximal end, a distal end and a clamping lumen extending from the proximal end to the distal end of the clamping part, wherein the sideways extension is attached to the clamping part in a non-detachable manner, preferably integrally formed with the clamping part, and wherein the clamping part is configured for clamping onto or around the body of the surgical guide tube. Herein, the method may further comprise the step of fixing the vascular graft to the body of the surgical guide tube by clamping the clamping part onto the vascular graft into which the surgical guide tube has been inserted. This has the further advantage that the sideways extension can be positioned more individually.

Therein, the clamping part may reduce the diameter of the guide lumen, i.e. narrow a cross-section of the guide lumen, when clamped onto the vascular graft into which the surgical guide tube is inserted. In particular, the clamping part may be clamped onto the vascular graft at a position of the surgical guide tube between two neighbouring bumps, between a proximal bulge and a bump which is located closest to the proximal bulge, or between a distal bulge and a bump which is located closest to the distal bulge which may be provided on the surgical guide tube, thereby fixing the medical device inside the guide lumen of the surgical guide tube such that longitudinal movement of the medical device inside the guide lumen is prevented.

Alternatively or additionally, the method may further comprise the step of fixing the vascular graft to the body of the surgical guide tube, which has previously been inserted into the graft, by applying at least one of at least one suture and at least one fixing clamp onto the graft into which the surgical guide tube is inserted. In particular, the vascular graft may be fixed to the body of the surgical guide tube at a longitudinal position of the surgical guide tube between two neighbouring bumps, between a proximal bulge and a bump which is located closest to the proximal bulge, or between a distal bulge and a bump which is located closest to the distal bulge provided on the surgical guide tube. Therein, the suture or fixing clamp may reduce the diameter of the graft lumen, i.e. narrow a cross-section of the graft lumen, when applied to the graft into which the surgical guide tube is inserted. In particular, the diameter of the graft lumen may be narrowed to a reduced diameter which is smaller than an outer diameter of two neighbouring bumps, a proximal bulge and a bump which is located closest to the proximal bulge, or a distal bulge and a bump which is located closest to the distal bulge provided on the surgical guide tube, thereby reducing, preferably preventing, the risk of longitudinal movement of the surgical guide tube inside the graft lumen.

Preferably, the fixing clamp may reduce the diameter of the guide lumen, i.e. narrow a cross-section of the guide lumen, when clamped onto the vascular graft into which the surgical guide tube is inserted. Preferably, the fixing clamp is placed at a longitudinal position of the surgical guide tube between two neighbouring bumps, between a proximal bulge and a bump which is located closest to the proximal bulge, or between a distal bulge and a bump which is located closest to the distal bulge provided on the surgical guide tube, thereby fixing the medical device inside the guide lumen of the surgical guide tube such that longitudinal movement of the medical device inside the guide lumen is prevented.

In a preferred embodiment, the method may comprise the step of fixing a fixator to the patient's skin such that the medical device is held to or against the patient's body. The fixator has a central body and two fixator wings extending from the central body in opposite directions, and each fixator wing has a planar surface, the planar surfaces facing in a common direction. This provides an additional fixation of the medical device to the patient's body. Therein, each of the two fixator wings may comprise at least one aperture, preferably two or more apertures, and the method may comprise the step of inserting a suture or clamp through said aperture such that the fixator wings are attached, i.e. sutured or clamped, to the patient's skin. Alternatively or additionally, the planar surfaces of the fixator wings may at least partially or completely be covered with an adhesive, and the method may further comprise the step of sticking the planar surfaces to the patient's skin.

Further preferably, the central body of the fixator may have a proximal end, a distal end and a fixator lumen extending from the proximal end to the distal end of the central body, wherein the method may further comprise the step of inserting the medical device into and through the fixator lumen. Preferably, the central body of the fixator is slidably mounted on the medical device.

Preferably, a tubular extension having an extension lumen is attached to, preferably integrally formed with, the distal end of the central body of the fixator, wherein the method may further comprise the step of inserting the medical device into and through the extension lumen. Preferably, the tubular extension is slidably mounted on the medical device.

In particular, a repositioning unit may be provided having a proximal end, a distal end and a repositioning lumen extending from the proximal end to the distal end of the repositioning unit, wherein the method may further comprise the step of inserting the medical device into and through the repositioning lumen. The repositioning unit may further comprise at least one button, or lever, which is accessible from outside the repositioning unit and which preferably protrudes to or extends through the circumferential outer surface of the repositioning unit. The method may further comprise the step of using the button or lever, in a first state, to hold the medical device in its longitudinal position relative to the repositioning lumen and, in a second state, release the medical device upon actuation of the button or lever so as to allow movement of the medical device relative to the repositioning lumen. Preferably, the repositioning unit is attached to the proximal end of the central body of the fixator.

In a preferred embodiment, the method may further comprise the step of attaching the fixator to a location on the outer surface of the patient's skin such that a section of the medical device, which extends from the fixator to the surgical guide tube, may be arranged in a C-curve on the patient's skin. This arrangement provides the advantage that movement of the patient's skin, in particular in the shoulder region, can be absorbed by the C-curve, i.e. the C-curve section of the medical device can be compressed or stretched. A pulling or pushing force on the anastomosis, the intravascular blood pump or both can therefore be prevented.

Further preferably, the central body of the fixator may form an arched, i.e. convex, bridge, and the method may further comprise the step of applying the fixator over the medical device and onto the outer surface of the patient's skin such that the medical device passes between the central body of the fixator and the patient's skin, i.e. underneath the bridge. This provides a further fixation of the medical device to the patient's body.

Preferably, the medical device is inserted into and through a fixation graft which may comprise a velour, such as Dacron, on its outer surface configured for skin or tissue to grow into, and the method may comprise the step of implanting the fixation graft under the patient's skin such that a section of the medical device between the surgical guide tube and the fixator extends under and along the skin and tissue can grow into the velour. In this way, a further fixation under the patient's skin can be achieved by way of the velour, in addition to the surgical guide tube which is fixed to the fascia under the patient's skin, thereby providing a further means for fixing the medical device to the patient's body.

In a preferred embodiment, a tubular extension having an extension lumen may be attached to, preferably integrally formed with, the surgical guide tube at the proximal end of the surgical guide tube, and the method may comprise the step of inserting the medical device into and through the extension lumen. The medical device is additionally held and guided by the tubular extension of the surgical guide tube.

Further, a connecting portion having a connecting lumen may connect a proximal end of the tubular extension with a distal end of the above-described repositioning unit, wherein the medical device may be inserted into and through the connecting lumen. Therein, the connecting portion may have along its length a section with a reduced outer diameter, said section being positioned between the repositioning unit and the tubular extension so as to form a waist portion between the repositioning unit and the tubular extension. Therein, the central body of the fixator may form an arched, i.e. convex, bridge, and the method may comprise the step of applying the fixator over the connecting portion and onto the patient's skin such that only the waist portion of the connecting portion passes between the arched bridge of the fixator's central body and the outer surface of the patient's skin, i.e. the waist portion of the connecting portion passes underneath the bridge. Preferably, the waist portion has a proximal end and a distal end and the waist portion can move under the bridge along a direction, which is the direction of the longitudinal length of the connecting lumen, wherein movement of the waist portion is stopped at the distal end and at the proximal end of the waist portion, respectively. Thereby, the waist portion provides a range of movement for the connecting portion within which the connecting portion can be pushed back and forth while at the same time being attached to the patient's skin.

In a preferred embodiment, the surgical guide tube including the sideways extension is part of a clamping unit as described above with reference to the fourth aspect of the present disclosure. Therein, the central body of the fixator may form an arched, i.e. convex, bridge, and the method may comprise the step of applying the fixator over the waist portion of the clamping unit onto the patient's skin such that only the waist portion passes between the central body of the fixator and the outer surface of the patient's skin, i.e. the waist portion passes underneath the bridge. Preferably, the waist portion has a proximal end and a distal end and the waist portion can move under the bridge along a direction, which is the direction of the longitudinal length of the lumen of the clamping unit, wherein movement of the waist portion is stopped at the distal end and at the proximal end of the waist portion, respectively. Thereby, the waist portion provides a range of movement for the clamping unit's handling portion within which the handling portion can be pushed back and forth while at the same time being attached to the patient's skin.

An exemplary method of providing a vascular access to a patient's vessel, in particular a blood vessel and preferably at least one of the arteria axillaris and the arteria subclavia, and inserting an intravascular blood pump comprising a catheter at its proximal end into the patient's vascular system, and attaching the catheter to the patient's body, comprises the following steps:
suturing a vascular graft to the patient's vessel by performing an anastomosis, thereby providing vascular access to the vessel,
introducing the intravascular blood pump into and through the vascular graft into the vessel and advancing the intravascular blood pump to its intended position inside the patient's body, i.e. across the aortic valve in the left ventricle of the patient's heart,
sliding a surgical guide tube, which is slidably mounted on the outer surface of the catheter, along the catheter into the surgical graft,
fixing the surgical graft to the surgical guide tube inserted therein, e.g. by sutures or by means of a clamp,
fixing a sideways extension of the surgical guide tube to a deep fascia of the patient between the vessel and the patient's skin, in particular to a deep fascia which extends in the vicinity of the vessel,
fixing the catheter, directly or indirectly, to an outer surface of the patient's skin.

In said exemplary method, any of the above-described embodiments of the surgical guide tube, the kit, the system, and the clamping unit can be applied.

A different method of stabilizing a catheter-based medical device in a vessel of a patient, according to the present disclosure, does not require the use of the surgical guide tube. According to this method, the patient's skin is opened to get access to the vessel, a graft is connected to the vessel by anastomosis, and the medical device is inserted through the graft into the vessel so that the catheter extends through the graft out of the patient. Then, the graft is twisted so as to reduce its inner diameter, thereby clamping the catheter inside the graft. This may be further improved as described below by additional means so that torsion forces are not transferred to the anastomosis.

Once the graft has been twisted, it may be held in the twisted state so that it cannot untwist. This may be done by temporarily clamping a proximal part of the twisted graft against the catheter using a clamp which acts on the graft from the outside thereof. Next—or without the additional clamping step—the twisted graft may be sutured to hold it constantly in the twisted state, i.e. the twisted graft is sutured to itself in order to conserve the twisted structure. In addition, the twisted graft may be shortened to a desired length either prior to or, preferably, after suturing it.

The step of connecting the graft to the vessel by anastomosis may include suturing the graft to the vessel thereby creating a seam between the graft and the vessel, and the seam is preferably enforced with glue.

As mentioned, the medical device may be an intravascular blood pump. In such a case, the step of introducing the medical device into the vessel includes advancing the intravascular blood pump into the patient's blood vessel, e. g. the arteria axillaris or arteria subclavia, until the pumping device of the intravascular blood pump reaches into the patient's heart.

Preferably, the medical device comprises a plug which is slidable along the catheter. Once the medical device is placed at the target location inside the patient, the slidable plug is arranged on the catheter inside the graft so that it is in close proximity to the vessel. One purpose of the plug is to seal the graft against the catheter so as to provide haemostasis. Due to the plug being slidable on the catheter, it can be loosely placed on the catheter so that, when the medical device is introduced through the graft into the vessel towards its final position, the plug slides backwards on the catheter. The slidable plug preferably has a closed form. The closed form may be a closed cylindrical form and preferably the outer form of the slidable plug corresponds to a circular cylinder, thereby stabilizing the position of the plug inside the graft. Further, the slidable plug may have a through hole through which the catheter extends, said through hole having a hexagonal shape in cross section and preferably the form of a hexagonal cylinder. The hexagonal shape can help to reduce friction while the plug slides along the catheter. The slidable plug may be made of silicone rubber. The slidable plug may have a strength of 50 shore A.

Preferably, prior to arranging the plug inside the graft, a slotted shim is placed around the graft at a location distal of where the slidable plug is intended to be arranged inside the graft. The slotted shim is inelastic and comprises a central through hole from which a slot extends radially outwards so as to form an aperture in the radial direction of the shim. In order to arrange the shim around the graft with the catheter inserted therein, the graft can be passed through the slot to the central through hole of the shim. The inner diameter of the central through hole is smaller than the outer diameter of the slidable plug so that the plug cannot be pushed past the shim and into the vessel, but abuts the shim when pushed distally. The slotted shim may further comprise an eyelet arranged at its radially outer circumference. The eyelet serves as a fixing means for fixing the shim to a fascia of the patient, e. g. by suturing. The slotted shim can be easily grasped and held by the surgeon at the time when the graft is being twisted. During the twisting movement, the shim and the plug serve as a twisting stop so that the torsion forces created by the twisting do not reach the anastomosis and, thus, cannot negatively affect the anastomosis seam.

In other words, the step of twisting the graft so as to reduce its inner diameter preferably includes holding the slotted shim, thereby facilitating the twisting step. The shim is preferably made of PEEK.

Preferably, immediately prior to the twisting step, the position of the medical device inside the patient's body is corrected or finalized. This is beneficial, because sliding the plug to its intended position inside the graft might lead to an unwanted displace-ment of the medical device so that the position of the medical device needs a little correction after the plug is properly placed.

When the graft is properly fixed to the catheter by the clamping forces generated by the twisted graft, the medical device can no longer move forwards or backwards inside the vessel. Therefore, the twisted graft may finally be pushed into the patient's body while the catheter extends through the twisted graft out of the patient's body, and the opened skin may then be closed around the catheter.

A sleeve, such as a tubular part made of polytetrafluoroethylene (PTFE), may be provided around the twisted graft in order to prevent ingrowth of tissue into the graft. That is, the graft is usually made of Dacron® or a similar material, and it must be ensured that the graft can be untwisted without efforts when the medical device is to be removed from the vessel. Ingrowth of tissue in the graft would hinder untwisting.

According to another aspect of the present disclosure, a kit for stabilizing a medical device comprising a catheter is provided. The kit comprises a vascular graft through which the medical device can be introduced and through which the catheter can extend. The graft can be twistable so as to reduce its inner diameter in order to clamp the catheter inside the graft. The kit further comprises a plug which is configured to be slidable along the catheter and which is insertable into the graft, preferably while the plug is placed on the catheter. The graft can be sutured to itself, in particular in its twisted state. Preferably, the graft is knitted, warp-knitted, knotted, woven or a nonwoven fabric which facilitates suturing of the graft.

The plug may have a closed form, preferably a closed cylindrical form. E. g. the outer form of the slidable plug corresponds to a circular cylinder. Further, the plug may have a through hole through which the catheter can extend. Such through hole preferably has a hexagonal shape in cross section and is particularly in the form of a hexagonal cylinder. The plug can be made or silicone rubber and may have a strength of 50 shore A.

Particularly, the kit further comprises a slotted shim which is configured for being placed around the graft while the catheter is inserted in the graft and which may be made of PEEK. The slotted shim is preferably not elastic. The slotted shim preferably comprises a radially extending slot through which the graft can be passed towards the centre of the shim while the catheter extends through the graft. Further, the shim may be provided with an eyelet by which the shim can be fixed to a patient's fascia.

Preferably, an inner diameter of the slotted shim is smaller than an outer diameter of the plug, so that the plug cannot pass through the shim. Also preferably, the inner diameter of the slotted shim is larger than an outer diameter of the medical device, so that the medical device can be passed through the shim.

The kit may further include an intravascular blood pump as the medical device. Further, the kit may comprise a clamp which is configured for clamping a proximal part of the twisted graft against the catheter.

While the above disclosure is described in connection with a blood pump that is positioned within a heart, in particular the left ventricle, other applications are possible and not excluded from the scope of the present disclosure. For example, the entire present disclosure can be applied to the stabilization of the position of a blood pump for right-side support (right ventricle) or other portions of the vasculature, e.g. near the kidneys in case that a blood pump is arranged to promote kidney perfusion. Further, the entire present disclosure, at least aspects thereof, can be applied to any kind of catheter-based devices other than blood pumps, which may need to be positioned at a particular location in the vasculature. Moreover, while it is described that the medical device can be introduced into the aorta via the arteria subclavia or arteria axillaris, the disclosure is not restricted to an access via the arteria subclavia or arteria axillaris. It is also within the disclosure to provide an access for a medical device via the aorta directly or to provide a suprasternal access. Further, femoral vessels may likewise be accessed. The entire disclosure is also applicable in pae-diatrics.

### DESCRIPTION OF THE FIGURES

Hereinafter, the present disclosure will be described by way of example with reference to the accompanying drawings. Therein are shown:

FIG. 1 a side view of a surgical guide tube, according to a first embodiment of the present disclosure, and a vascular graft which are attached to a patient's body;

FIG. 2 (a)-(d) different embodiments of a fixing clamp;

FIG. 3 a perspective view of a surgical guide tube according to a second embodiment of the present disclosure;

FIG. 4 a plan view of a clamping part;

FIG. 5 a side view of the surgical guide tube, according to the second embodiment of the present disclosure, and a vascular graft which are attached to a patient's body;

FIG. 6 a plan view of a system for fixing a medical device to a patient's body according to a first embodiment of the present disclosure;

FIG. 7 a plan view of a system for fixing a medical device to a patient's body according to a second embodiment of the present disclosure;

FIG. 8 a sectional view of the system according to the second embodiment of the present disclosure;

FIG. 9 a plan view of a system for fixing a medical device to a patient's body, according to a third embodiment of the present disclosure, which is attached to a patient's body;

FIG. 10 an enlarged side view of section X of FIG. 9;

FIG. 11 a partial sectional plan view of a clamping unit in a first state;

FIG. 12 a partial sectional plan view of the clamping unit of FIG. 11 in a second state; and

FIG. 13 a vascular graft twisted on a catheter.

FIG. 14A a plan view of an elastic open ring

FIG. 14B a sectional view of the elastic open ring of FIG. 14A along line XIV

FIG. 15A a three dimensional view of a plug

FIG. 15B a cross sectional view of the plug of FIG. 15A

The drawings are not to scale and should not be constructed as limiting the present disclosure in any manner.

### DETAILED DESCRIPTION

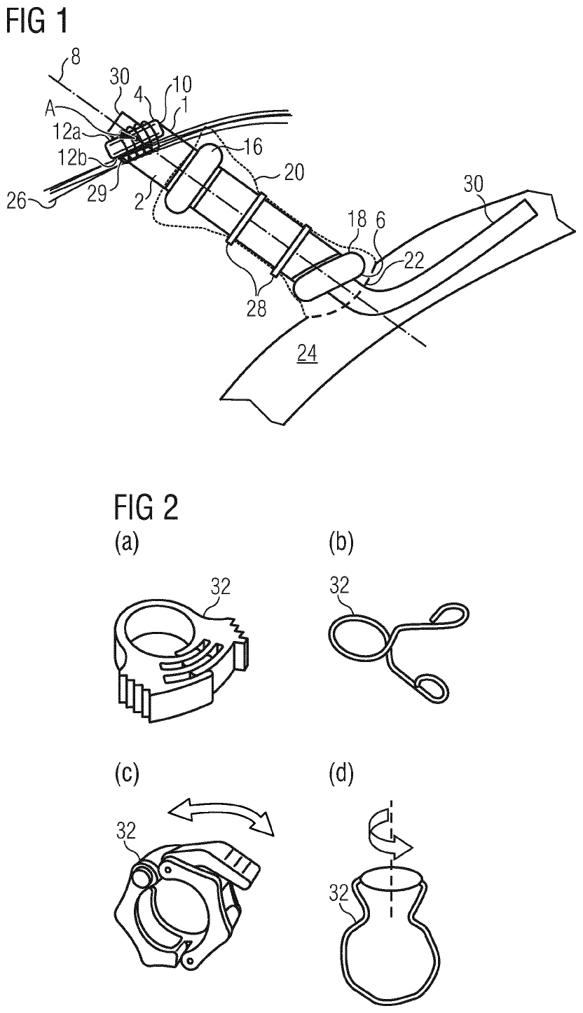

FIG. 1 shows a side view of a surgical guide tube 1 according to a first embodiment of the surgical guide tube. The surgical guide tube 1 comprises a tubular body 2 having a proximal end 4, a distal end 6 and a guide lumen extending from the proximal end 4 to the distal end 6, the guide lumen having a longitudinal axis 8. The surgical guide tube 1 comprises a sideways extension 10 which extends sideways from the tubular body 2 and defines planar surfaces 12a, 12b, of which the planar surface 12a is a proximal or front planar surface and the planar surface 12b is a distal or back planar surface. The sideways extension 10 forms a wing which extends in a radial direction relative to the longitudinal axis 8 of the guide lumen. A further wing forming a further sideways extension (not shown in FIG. 1) is provided on the tubular body 2 radially opposite to the sideways extension 10 shown in FIG. 1. Thus, the two wings extend in opposite radial directions. The planar surfaces 12a, 12b of the sideways extension 10 are parallel to each other and are inclined relative to the longitudinal axis 8 of the guide lumen by an inclination angle of about 45°. Each planar surface 12a, 12b comprises an axis which extends orthogonally through the longitudinal axis 8 of the guide lumen and serves as a virtual rotational axis A about which the planar surface 12a, 12b is rotated into said inclination angle.

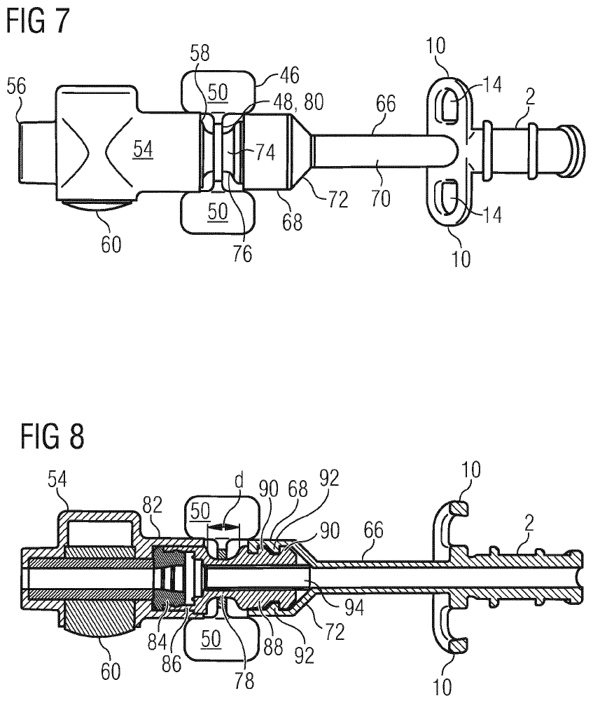

The sideways extension 10 (as well as the sideways extension on the radially opposite side of the tubular body 2) is integrally formed with the tubular body 2 and is located at the proximal end 4 of the tubular body 2. The sideways extension 10 (as well as the sideways extension on the radially opposite side of the tubular body 2) comprises an aperture 14 having a D-shaped transverse cross-section and has a longitudinal axis extending in a substantially vertical direction relative to the planar surface 12a, as e.g. shown in the embodiment of FIG. 7.

Distal of the sideways extension 10, there is provided a circumferential bump 16 on the tubular body 2. The circumferential bump 16 extends radially from the tubular body 2 and has a cross-section in the shape of a circular ring. A cross-sectional plane of the circumferential bump 16 extends orthogonally to the longitudinal axis 8 of the guide lumen. At the distal end 6 of the tubular body 2, there is provided a distal bulge 18 surrounding the tubular body 2 and having a cross-section in the shape of a circular ring. The circumference of the distal end 6 of the surgical guide tube 1, i.e. the distal end of the distal bulge 18, lies in a plane which is inclined relative to the longitudinal axis 8 of the guide lumen by about 45°. This plane is inclined in the same direction and by the same inclination angle as the planar surfaces 12a, 12b, i.e. said plane and the planar surfaces 12a, 12b are parallel to each other. The tubular body 2 has a constant inner diameter, i.e. its guide lumen has a constant diameter.

The arrangement is shown with a distal end portion of the surgical guide tube 1 inserted in a vascular graft 20. Specifically, the surgical guide tube 1 is inserted in the graft 20 up to and including the circumferential bump 16. At a distal end 22 of the graft 20, the graft 20 is attached to a vessel 24 of a patient, e.g. by anastomosis. Thereby, the graft 20 provides access to the vessel 24 which is a blood vessel, e.g. the arteria axillaris or the arteria subclavia. With the surgical guide tube 1 being inserted in the graft 20, the circumferential bump 16 and the distal bulge 18 provide a light seal to reduce and ideally prevent blood loss through the gap between the tubular body 2 and an inner wall of the graft 20. The inner wall of the graft 20 is then completely sealed against the tubular body 2, i.e. the gap between the inner wall of the graft 20 and the tubular body 2 is completely closed, by means of two sutures 28 which fixate the graft 20 to the surgical guide tube 1. Alternatively, the inner wall of the graft 20 can be completely sealed against the tubular body 2 by more or fewer sutures or by means of a fixing clamp, which will be described later.

The sideways extension 10 is fixated on a deep fascia 26 of the patient which extends between the access vessel 24 and the patient's skin (not shown). Preferably, a deep fascia is chosen as a fixing point which lies in the vicinity of the accessed vessel 24. For this purpose, the sideways extension 10 is sutured to the deep fascia 26 with the distal planar surface 12b contacting the deep fascia 26 by means of sutures 29 passing through the aperture 14, through or around the deep fascia 26 and further around the sideways extension 10. Alternatively, the sideways extension 10 may be clamped or glued to the deep fascia 26. In the same way, it is likewise possible to fixate the fascia 26 to the sideways extension 10 with the proximal planar surface 12a contacting the deep fascia 26.

Through the guide lumen, a catheter 30, e.g. a catheter which is attached to a proximal end of an intravascular blood pump (not shown), is inserted in the accessed vessel 24. The surgical guide tube 1 is slidable on the catheter 30, i.e. the catheter 30 and the surgical guide tube 1 can move relative to each other in a longitudinal direction. After the surgical guide tube 1 has been fixed to the vascular graft 20 by means of the sutures 28, the catheter 30 can still move back and forth longitudinally inside the guide lumen of the surgical guide tube 1. This allows for later replacement of the intravascular blood pump.

Instead of the sutures 28, the graft 20 can be fixed to the surgical guide tube 1 by means of a fixing clamp 32 as shown by the embodiments (a) to (d) depicted in FIG. 2. Therein, embodiment (a) is a fixing clamp in the form of a cable tie, embodiment (b) is a clip or spring, embodiment (c) is a fixing clamp with a flap lock, and embodiment (d) is a tubing clip. Once the surgical guide tube 1 has been inserted into the vascular graft 20, with the catheter 30 being inserted through the guide lumen of the surgical guide tube 1, the fixing clamp 32 is clamped onto the graft 20, the fixing clamp 32 reducing the diameter of guide lumen such that the catheter 30 is fixated inside the guide lumen. This means that longitudinal movement of the catheter 30 inside the surgical guide tube 1 is prevented by means of the fixing clamp 32 clamped onto the vascular graft 20. Relative longitudinal movement of the surgical guide tube 1 and the vascular graft 20 is prevented by the fixing clamp 32 as well. Therefore, the catheter 30 is fixedly held in the intended position and dislocation of the intravascular blood pump can be avoided.

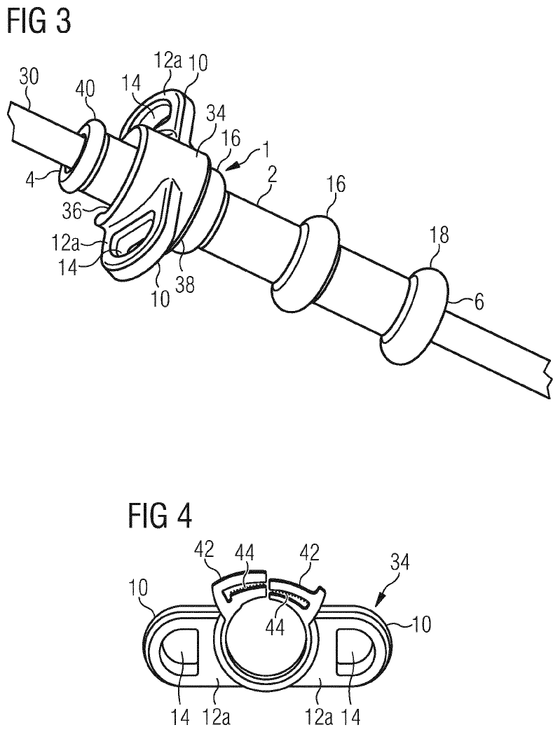

FIG. 3 shows a perspective view of a second embodiment of a surgical guide tube 1. The surgical guide tube 1 of the second embodiment differs from the surgical guide tube 1 of the first embodiment in the following features. The sideways extension 10 is provided on a separate clamping part 34 which can be clamped onto the tubular body 2 of the surgical guide tube 1. The clamping part 34 comprises a proximal end 36, a distal end 38 and a clamping lumen extending from the proximal end 36 to the distal end 38. The sideways extension 10 is integrally formed with the clamping part 34.

The planar surfaces 12a, 12b of the sideways extension 10 are inclined relative to a longitudinal axis of the clamping lumen by an inclination angle of about 45°. When the clamping part 34 is clamped onto the tubular body 2, the planar surfaces 12a, 12b are also inclined by an inclination angle of about 45° relative to the longitudinal axis of the guide lumen, i.e. the longitudinal axis of the clamping lumen and the longitudinal axis 8 of the guide lumen are in parallel, and may even coincide, when the clamping part 34 is clamped onto the surgical guide tube 1.

A proximal bulge 40 surrounds the tubular body 2 and forms a proximal end of the surgical guide tube 2. The proximal bulge 40 has a cross-section in the shape of a circular ring. Distal of the proximal bulge 40, there are provided two circumferential bumps 16 and the distal bulge 18. The proximal bulge 40, the circumferential bumps 16 and the distal bulge 18 are aligned in parallel to each other, and the circumferential bumps 16 and the distal bulge 18 have a same outer diameter, which diameter is larger than an outer diameter of the proximal bulge 40.

The proximal bulge 40, the circumferential bumps 16 and the distal bulge 18 are positioned along the longitudinal axis 8 of the guide lumen at mutual distances which are larger than a distance from the proximal end 36 of the clamping part 34 to the distal end 38 of the clamping part 34. Thereby, a clamping position of the clamping part 34 onto the tubular body 2 is variable. Precisely, the clamping part 34 can be clamped onto the tubular body 2 between neighbouring bumps or between a bump and a neighbouring bulge of the tubular body 2, i.e. between the proximal bulge 16 and the closest circumferential bump 16, between the two circumferential bumps 16, or between the distal bulge 18 and the closest circumferential bump 16.

FIG. 4 shows a plan view of the clamping part 34. The sideways extensions 10 are formed by two wings and comprise D-shaped apertures 14 which extend in a substantially vertical direction relative to the planar surfaces 12a, 12b. The clamping part 34 comprises two arms 42 extending in opposite circumferential directions. The arms 42 are each provided with an interlocking profile 44 and arranged such that a cross-section of the clamping part's clamping lumen is narrowed when the arms 42 are moved towards and past each other in the opposite circumferential directions. Once moved towards and past each other, the arms 42 interlock with each other via their interlocking profiles 44. The interlocking profiles 44 form a ratchet mechanism.

FIG. 5 shows a side view of the surgical guide tube 1 according to the second embodiment in a position where it is inserted in and fixed to the vascular graft 20, which is attached to the patient's accessed vessel 24. In contrast to the surgical guide tube 1 of the first embodiment, the tubular body 2 of the surgical guide tube 1 of the second embodiment is inserted in the vascular graft 20 up to and including the proximal bulge 16, i.e. up to and including the proximal end 4 of the surgical guide tube's tubular body 2. After the tubular body 2 has been inserted into the graft 20, with the catheter 30 being inserted through the guide lumen of the tubular body 2 into the accessed vessel 24, the clamping part 34 is clamped onto the vascular graft 20 at a position between the proximal bulge 40 and the closest circumferential bump 16. It is also possible to clamp the clamping part 34 at one of the other above-described clamping positions.

When the tubular body 2 has been inserted into the graft 20, with the catheter 30 being inserted through the guide lumen, and the clamping part 34 is clamped onto the graft 20, the clamping part 34 reduces the diameter of the guide lumen such that the catheter 30 is fixated inside the guide lumen of the surgical guide tube 1. This means that longitudinal movement of the catheter 30 inside the tubular body 2 is prevented by the clamping part 34 clamped onto the vascular graft 20. Longitudinal movement of the tubular body 2 inside the vascular graft 20 is also prevented by the clamping part 34. Therefore, the catheter 30 is fixedly held in the intended position and dislocation of the intravascular blood pump can be avoided. The sideways extension 10 can be attached to the deep fascia 26 in the same manner as described above with reference to the surgical guide tube 1 of the first embodiment.

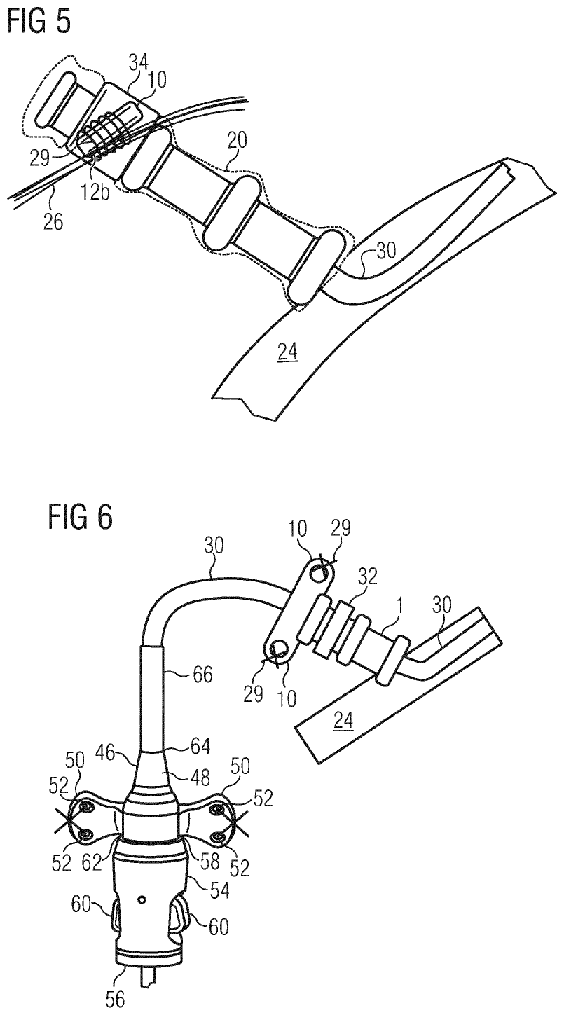

FIG. 6 shows a plan view of a system for fixing a medical device, e.g. a catheter, to a patient's body according to a first embodiment of the system. The system comprises a surgical guide tube 1 which can be the surgical guide tube 1 as already described above. However, the surgical guide tube 1 of this system for fixing a medical device can also have a different configuration, e.g. a different number of circumferential bumps 16. Specifically, the surgical guide tube 1 of this system for fixing a medical device has a sideways extension 10 with planar surfaces 12a, 12b which can be inclined relative to the longitudinal axis of the guide lumen by about 45°. However, this is not a necessary feature of this system for fixing a medical device and can be omitted without departing from the present disclosure. In particular, the planar surfaces 12a, 12b may have different inclination angles or no inclination at all. As in the above-described surgical guide tube 1, the sideways extension 10 is configured for fixing the surgical guide tube 1 to the patient's deep fascia (not shown in FIG. 6).

The system for fixing a medical device comprises a fixator 46 with a central body 48 and fixator wings 50 extending from the central body 48 in opposite directions. Each fixator wing 50 has a planar surface, and the planar surfaces of the two fixator wings 50 face in a common direction. The fixator wings 50 are configured for fixing the fixator 46 to an outer surface of the patient's skin and for holding the catheter 30 against the patient's body. For this purpose, the fixator wings 50 each comprise two apertures 52 through which sutures can be passed to suture the fixator wings 50 to the patient's skin. There may be more or fewer than two apertures 52 provided in the fixator wings 50.

The system for fixing a medical device comprises a repositioning unit 54 having a proximal end 56, a distal end 58 and a repositioning lumen extending from the proximal end 56 to the distal end 58. The repositioning unit 54 comprises two buttons 60 which are accessible from outside the repositioning unit 54. The two buttons 60 are designed to hold the catheter 30, in a first state, in its longitudinal position relative to the repositioning lumen 54 and release the catheter 30, in a second state, upon actuation of the buttons 60 so as to allow movement of the catheter 30 relative to the repositioning lumen 54.

The central body 48 of the fixator 46 has a proximal end 62, a distal end 64 and a fixator lumen extending from the proximal end 62 to the distal end 64 of the central body 46. A tubular extension 66 having an extension lumen is joined to the central body 48 at a distal end 64 of the central body 48. The repositioning unit 54 is attached to the proximal end 62 of the central body 48.

In use, the catheter 30 extends through the repositioning lumen, the fixator lumen, the extension lumen, and the guide lumen into the accessed vessel 24 of the patient, with the catheter 30 penetrating through the patient's skin. The vascular graft 20 (not shown in FIG. 6), the surgical guide tube 1 and the catheter 30 are fixated by the fixing clamp 32 in the above-described manner, and the sideways extension 10 is fixated onto the patient's deep fascia 26. It is also possible to apply the above-described second embodiment of the surgical guide tube 1 comprising the separate clamping part 34, wherein the clamping part 34 is used to fixate the vascular graft 20, the tubular body 2 and the catheter 30. Thereby, movement of the catheter 30 inside the vessel 24 can be prevented.

The tubular extension 66, the fixator 46 and the repositioning unit 54 are positioned proximal of the sideways extension 10. Therein, the tubular extension 66 is positioned at a distance to the sideways extension 10. The tubular extension 66 is placed such that it penetrates the patient's skin in order to reduce skin irritation. The catheter 30has a maximum stiffness of about 55D and should be as flexible as possible in order to reduce force transmission. The tubular extension 66 should have a stiffness of about 60A and should be configured as thin as possible in order to obtain a small diameter and reduce infection. The catheter 30 can be arranged so as to form a C-curve between the tubular extension 66 and the sideways extension 10. The C-curve of the catheter 30 can then absorb movement of the patient's body part, in particular movement of the skin in the shoulder region, to which the fixator 46 is attached, and a pulling force on the anastomosis can be prevented.

FIG. 7 shows a plan view of a system for fixing a medical device to a patient's body according to a second embodiment of the system. The second embodiment of the system for fixing a medical device comprises a surgical guide tube 1 which can comprise any one of the features of the first embodiment of the above-described surgical guide tube 1. However, the surgical guide tube 1 of this second embodiment of the system for fixing a medical device can also have a different configuration, e.g. a different number of circumferential bumps 16, which in the present case is two. Specifically, the surgical guide tube 1 of this second embodiment of the system for fixing a medical device has a sideways extension 10 with planar surfaces 12a, 12b which can be inclined relative to the longitudinal axis of the guide lumen by about 45°. This is, however, not a necessary feature and can be omitted from the system without departing from the present disclosure. The planar surfaces 12a, 12b can also have different inclination angles or no inclination at all. As in the first embodiment of the above-described surgical guide tube 1, the sideways extension 10 is configured for fixing the surgical guide tube 1 to the patient's deep fascia.

Like the first embodiment of the system for fixing a medical device, also this second embodiment of the system comprises a fixator 46 with a central body 48 and fixator wings 50 extending from the central body 48 in opposite directions. Each fixator wing 50 has a planar surface, with the planar surfaces of the two fixator wings 48 facing in a common direction. The fixator wings 50 are configured for fixing the central body 48 to the outer surface of the patient's skin and for holding the catheter 30 against the patient's body. For this purpose, the planar surfaces of the fixator wings 50 are each covered with an adhesive, e.g. like a plaster or adhesive tape, to stick the fixator wings 50 to the patient's skin.

Also, this second embodiment of the system for fixing a medical device comprises a repositioning unit 54 with a proximal end 56, a distal end 58 and a repositioning lumen extending from the proximal end 56 to the distal end 58. In contrast to the repositioning unit 54 of the first embodiment of the system for fixing a medical device, the repositioning unit 54 of this second embodiment of the system for fixing a medical device comprises only one button 60 for holding the catheter 30 in its longitudinal position relative to the repositioning lumen 54 in a first state and for releasing the catheter 30, in a second state, upon actuation of the button 60 so as to allow movement of the catheter 30 relative to the repositioning lumen 54.

The surgical guide tube 1 comprises a tubular extension 66 provided proximal of the sideways extension 10, both of which are integrally formed with the tubular body 2. At a proximal end of the tubular extension 66, the tubular extension 66 comprises a sleeve portion 68 with an enlarged diameter as compared to a distal end of the tubular extension 66 which is joined to the sideways extension 10. A distal portion 70 of the tubular extension 66, which has a constant diameter corresponding to the diameter at the distal end of the tubular extension 66, merges into a tapering portion 72 which connects the distal portion 70 of the tubular extension 66 with the sleeve portion 68.

A connecting portion 74 connects the sleeve portion 68 with the repositioning unit 54. While the distal end 58 of the repositioning unit 54 and a proximal end of the sleeve portion 68 have substantially the same diameter, the outermost diameter of the connecting portion 74 is smaller than this. The connecting portion 74 comprises a waist portion 76 within which the diameter of the connecting portion 74 is further reduced. At the distal end of the waist portion 76 and at the proximal end of the waist portion 76, the waist portion 76 tapers towards a central region 78 with a constant diameter, which is the smallest diameter of the waist portion 76.

The fixator 46 has a central body 48 in the form of an arched bridge 80, or bracket, connecting the two fixator wings 50. A width of the bridge 80 is small as compared to the width of the fixator wings 50, and the bridge 80 is adapted to pass over the waist portion 76 such that the waist portion 76 can move longitudinally under the bridge 80. The length of the central region 78 corresponds to approximately the double width of the bridge 80, and a proximal end of the waist portion 76 and a distal end of the waist portion 76 limit a span d of longitudinal movement of the waist portion 76 under the bridge 80 (see FIG. 8).

As can be seen in the sectional view of FIG. 8, the repositioning unit 54, the connecting portion 74 and the tubular extension 66 are connected via a plug connection. For this purpose, the repositioning unit 54 provides a radially outer guide sleeve 82 as well as a sealing 84 located inside the guide sleeve 82. The connecting portion 74 has a proximal sleeve 86 which is adapted to radially fit between the guide sleeve 82 and the sealing 84 in order to engage the sealing 84 in a non-detachable manner. A distal portion 88 of the connecting portion 74 is provided with a radially outer engagement structure 90, and the sleeve portion 68 of the tubular extension 66 is provided with a radially inner engagement structure 92. When the connecting portion 74 is plugged into the sleeve portion 68, the engagement structures 90, 92 engage in a non-detachable manner. A guide tube 94 is provided which is connected with the sealing 84. The guide tube 94 extends through the waist portion 76 and the distal portion 88 of the connecting portion 74 and forms the connecting lumen through which the medical device can be inserted. When the connecting portion 74 is plugged into the sleeve portion 68 of the tubular extension 66, the guide tube 94 of the connecting portion 74 abuts distally against the tapering portion 72 of the tubular extension 66 such that the distal end of the guide tube 94 provides a sealing function between the sleeve portion 68 and the distal portion 88 of the of the connecting portion 74.

In use, the catheter 30 (not shown in FIGS. 7 and 8) extends through the repositioning lumen, the connecting lumen, and the guide lumen into the accessed vessel 24 of the patient. The vascular graft 20 (not shown in FIGS. 7 and 8) and the surgical guide tube 1 are connected by sutures 28 (not shown in FIGS. 7 and 8) in the above-described manner (with reference to the first embodiment of the surgical guide tube 1) and the sideways extension 10 is fixated onto the patient's deep fascia 26. The tubular extension 66 penetrates through the patient's skin. Due to this configuration, the catheter 30 is fixedly positioned by the repositioning unit 54, which prevents unintended relocation of the catheter 30 and the intravascular blood pump inside the patient's vascular system, while the pump is still replaceable. Further, movement of the skin in the application region of the fixator wings 50, e.g. the shoulder region, is possible within the span d of longitudinal movement of the waist portion 76, i.e. a pushing or pulling force on the anastomosis can be reduced.

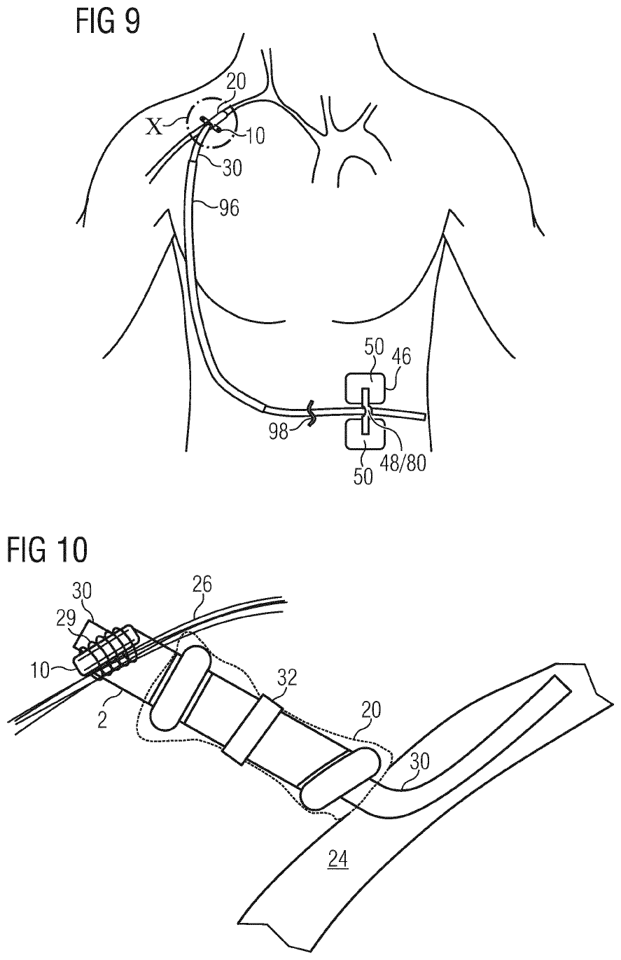

FIG. 9 shows a third embodiment of a system for fixing a medical device to a patient's body. Like the first embodiment of the system for fixing a medical device, this third embodiment of the system comprises a surgical guide tube 1 which can comprise any one of the features of the first or second embodiment of the surgical guide tube 1 described above. However, the surgical guide tube 1 of this third embodiment of the system for fixing a medical device can also have a different configuration. The surgical guide tube 1 of this third embodiment of the system for fixing a medical device has a sideways extension 10 with planar surfaces 12a, 12b which are inclined relative to the longitudinal axis of the guide lumen by about 45°. However, this is not a necessary feature and can be omitted from the system without departing from the present disclosure. The planar surfaces 12a, 12b can also have different inclination angles or no inclination at all. As in the first and second embodiment of the above-described surgical guide tube 1, the sideways extension 10 is configured for fixing the surgical guide tube 1 to the patient's deep fascia.

Like the second embodiment of the system for fixing a medical device, also this third embodiment of the system comprises a fixator 46 with a central body 48 and fixator wings 50 extending from the central body 48 in opposite directions. Each fixator wing 50 has a planar surface, with the planar surfaces of the two fixator wings 50 facing in a common direction. The fixator wings 50 are configured for fixing the central body 48 of the fixator 46 to the outer surface of the patient's skin and for holding the catheter 30 against the patient's body. For this purpose, the planar surfaces of the fixator wings 50 are each covered with an adhesive, e.g. like a plaster or adhesive tape, to stick the fixator wings 50 to the patient's skin.

The third embodiment of the system for fixing a medical device comprises a fixation graft 96 for implantation under the patient's skin and insertion of the medical device therethrough. On its outer surface, the fixation graft 96 is provided with a velour to enhance ingrowth of the fixator graft 96 into the tissue under the skin

In use, the catheter 30 penetrates through the patient's skin at a point of skin access 98 located proximal of a proximal end of the fixation graft 96, extends under the patient's skin through the fixation graft 96 and further through the surgical guide tube 1 into the vessel 24. The vascular graft 20 (shown in the enlarged view of section X in FIG. 10), the surgical guide tube 1 and the catheter 30 are fixated by the fixing clamp 32 in the above-described manner (see e.g. FIG. 2), and the sideways extension 10 is fixated on the patient's deep fascia 24. It is also possible to apply the second embodiment of the surgical guide tube 1 comprising the separate clamping part 34, wherein the clamping part 34 is used to fixate the graft 20, the tubular body 2 and the catheter 30. Due to this configuration, longitudinal movement of the catheter 30 inside the vessel 24 is prevented and dislocation of the intravascular blood pump is avoided.

The fixator 46 has a central body 48 in the form of an arched bridge 80, or bracket, connecting the two fixator wings 50. The bridge 80 is adapted to pass over the catheter 30 on an outer surface of the skin such that a part of the catheter 30 which extends on an outer surface of the skin proximal of the point of skin access 98 can move longitudinally under the bridge 80.

The third embodiment of the system for fixing a medical device is particularly suitable for long-term fixation of the medical device, which in the present case is the catheter 30 and the intravascular blood pump. When the medical device shall be removed from the patient's body, the vascular graft 20 and the fixing clamp 32 (or, in case of the second embodiment of the surgical guide tube 1, the separate clamping part 34) are exposed at the patient's axillary, and the catheter 30 is then cut off at the axillary and at the point of skin access 98. The part of the catheter 30 between the cutting point at the axillary and the point of skin access 98 remains in the patient's body. The vascular graft 20 and the fixing clamp 32 (or, in case of the second embodiment of the surgical guide tube 1, the separate clamping part 34) are removed from the patient's body.

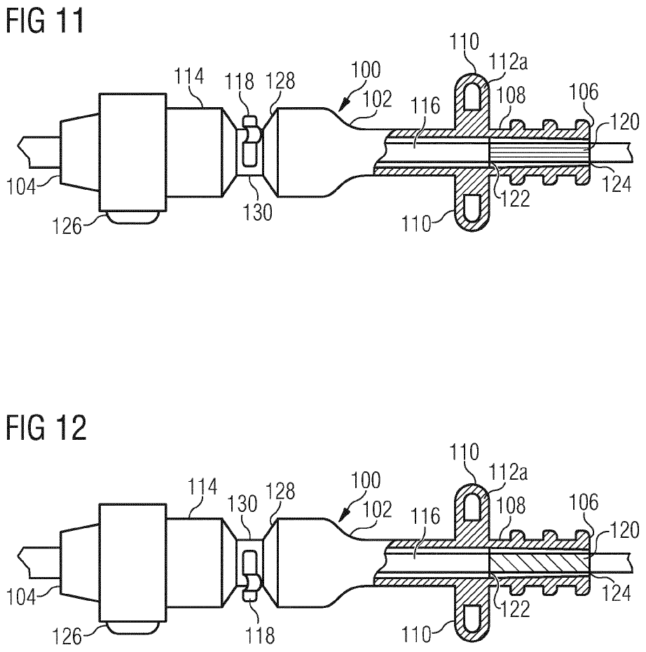

FIG. 11 shows a partial sectional view of a clamping unit 100 for fixation of a medical device to a patient's body in a first state. The clamping unit 100 has a tubular body 102 with a proximal end 104, a distal end 106 and a lumen for insertion of the medical device therethrough, the lumen extending from the proximal end 104 to the distal end 106. The tubular body 102 comprises a surgical guide tube portion 108 at the distal end 106 of the tubular body 102 and two sideways extensions 110 provided proximal of the surgical guide tube portion 108. Each sideways extension 110 defines a proximal planar surface 112a and a distal planar surface 112b (not shown). The tubular body 102 further comprises a handling portion 114 provided proximal of the sideways extension 110 and a rotatable tube 116 configured for insertion of the medical device therethrough. The rotatable tube 116 is provided inside the inner lumen of the tubular body 102 and operatively connected to a lever 118 which is accessible from outside the handling portion 114. The lever 118 protrudes through an outer surface of the handling portion 114, and actuation of the lever 118 causes rotation of the rotatable tube 116 about its longitudinal axis in a non-twisting manner.

Further, the tubular body 102 comprises a flexible tubular foil portion 120 which is configured for insertion of the medical device therethrough. The tubular foil portion 120 is provided inside the inner lumen and fixed to the rotatable tube 116 at a proximal connecting portion 122 such that the tubular foil portion 120 and the rotatable tube 116 are fixed to each other in a non-rotatable manner at the proximal connecting portion 122. The tubular foil portion 120 is further fixed to the inner wall of the tubular body 102 (inside the lumen of the tubular body) at a distal connecting portion 124 such that the tubular foil portion 120 and the tubular body 102 are fixed to each other in a non-rotatable manner at the distal connecting portion 124.

Due to this configuration, the tubular foil portion 120 is caused to twist about its longitudinal axis when the rotatable tube 116 is caused to rotate in a first direction and untwist when the rotatable tube 116 is caused to rotate in a second direction opposite to the first direction after the rotatable tube 116 has been rotated in the first direction. FIG. 11 shows the state in which the tubular foil portion 120 is untwisted, and FIG. 12 shows the state in which the tubular foil portion 120 is twisted. To transfer the tubular foil portion 120 from the untwisted state to the twisted state, i.e. to cause the rotatable tube 116 to rotate in the first direction, the lever 118, which is connected to the rotatable tube 116, is moved from a first position shown in FIG. 11 (lever 118 turned up) to a second position shown in FIG. 12 (lever 118 turned down). In the same manner, the tubular foil portion 120 is transferred from the twisted state to the untwisted state, i.e. the rotatable tube 116 is rotated in the second direction, by moving the lever 118 from the second position (lever 118 turned down) back to the first position (lever 118 turned up).

The handling portion 114 further comprises a button 126 which is accessible from outside the handling portion 114 and which extends through the circumferential outer surface of the handling portion 114. The button 126 is configured, in a first state, for holding the medical device in position relative to the lumen of the tubular body 102 and, in a second state, for releasing the medical device upon actuation of the button 126 so as to allow longitudinal and rotational movement of the medical device relative to the inner lumen. However, the button 126 is not a necessary feature and can be omitted from the clamping unit 114 without departing from the present disclosure.

The handling portion 114 comprises along its length a section with a reduced outer diameter so as to form a waist portion 128. At a distal end of the waist portion 128 and at a proximal end of the waist portion 128, the waist portion 128 tapers towards a central region 130 with a constant diameter, which is the smallest diameter of the waist portion 128. The lever 118 is provided within the central region 130 of the waist portion 128 and does not extend radially beyond portions of the handling portion 114 adjacent to the waist portion 128.

The clamping unit 100 can be combined with the above-described fixator 46 of the second embodiment of the system for fixing a medical device. Therein, the fixator wings 50, which are covered with adhesive, can fixate the central body 48 of the fixator to the patient's skin and hold the handling portion 114 against the patient's body. The central body 48 in the form of an arched bridge 80 can pass over the waist portion 128 of the handling portion 114 such that the waist portion 128 can longitudinally move under the bridge 80. The longitudinal length of the central region 130 corresponds to approximately the double width of the bridge 80, and a proximal end of the waist portion 128 and a distal end of the waist portion 128 limit a span of longitudinal movement of the waist portion 128 under the bridge 80.

In use, the catheter 30 is inserted into the lumen of the tubular body 102 with the lever 118 in the first position (lever 118 turned up), i.e. with the tubular foil portion 120 in an untwisted state, and extends through the entire handling portion 114 and surgical guide tube portion 108. That is, the catheter 30 also extends through the lumens of the rotatable tube 116 and the tubular foil portion 120, into the accessed vessel 24 of the patient. The vascular graft 20 (not shown in FIGS. 11 and 12) and the surgical guide tube portion 108 are connected by sutures 28 (not shown in FIGS. 11 and 12) in the above-described manner (with reference to the first embodiment of the surgical guide tube 1), and the sideways extension 110 is fixated to the patient's deep fascia 26. The handling portion 114 penetrates through the patient's skin. Due to this configuration, the catheter 30 can be fixedly positioned inside the clamping unit 100 by moving the lever 118 to the second position (turning the lever 118 down), thereby twisting the tubular foil portion 120, which results in reducing the inner diameter of the tubular foil portion 120 and clamping of the catheter 30. Thereby, unintended relocation of the catheter 30 and intravascular blood pump inside the patient's vascular system can be prevented. Further, the catheter 30 can be released from clamping by moving the lever 118 back to the first position (turning the lever 118 up), thereby untwisting the tubular foil portion 120, which results in enlarging the inner diameter of the tubular foil portion 120 and unclamping of the catheter 30. Thereby, the catheter 30 and the intravascular blood pump can be re-located or replaced. Further, movement of the skin in the region where the fixator wings 50 are applied, e.g. the shoulder region, is possible within the span of longitudinal movement of the waist portion 128. This way, a pushing or pulling force on the anastomosis can be reduced even when the catheter 30 is clamped inside the tubular foil portion 120, i.e. with the lever 118 in the second position (lever 118 turned down).

A different method of stabilizing a catheter-based medical device in a vessel of a patient, so that it cannot move forward or backward inside the vessel when the patient moves, does not require the use of a surgical guide tube. The first method steps are the same as in the previously described embodiments. That is, in a first step the patient's skin is opened to get access to the vessel, next a vascular graft is connected to the vessel by anastomosis, wherein the seam generated by suturing the graft to the vessel may be enforced with a glue, and then the medical device is inserted through the graft into the vessel so that the catheter extends through the graft out of the patient with its proximal end. However, according to this different method, instead of employing a surgical guide tube to stabilize the catheter, the graft is twisted so as to reduce its inner diameter, thereby clamping the catheter inside the graft that is fixed to the vessel.

More specifically, the medical device comprises a plug which is mounted on the catheter, so that the plug is slidable along the catheter. The slidable plug may have a closed form, such as a closed cylindrical form, in particular an outer form corresponding to a circular cylinder, and may be made of silicone rubber. Once the medical device, such as an intravascular blood pump, is placed at its target location inside the patient, the slidable plug arranged on the catheter is pushed into the graft to a position close to the anastomosis. By additional means described below, the slidable plug is held back in front of the vessel in close proximity thereto. To facilitate the insertion of the plug into the graft, the outer diameter of the plug may be smaller than the inner diameter of the graft. Additionally or alternatively, the graft may have elastic properties so that also a plug the outer diameter of which equals the inner diameter of the graft or even exceeds the inner diameter of the graft can still be pushed into the graft. In the latter case, tension is applied to the graft in radial and circumferential direction of the graft where the plug is positioned. Due to this tension and the elastic properties of the graft, a clamping force is generated which stabilizes the position of the plug inside the graft.

Prior to inserting the plug into the graft, a slotted shim, which is preferably not elastic and may be made of PEEK, is placed around the graft at a location distal of where the slidable plug is intended to be arranged inside the graft, thereby forming a stop for the slidable plug in the distal direction. The slotted shim is placed as close to the anastomosis as possible. The purpose of the slotted shim, in addition to serving as a sliding stop for the plug, is that it can be easily grasped and held by the surgeon at the time when the graft is being twisted. Further, the shim may be provided with an eyelet which protrudes radially outwards and by which the shim can be fixed, e. g. sutured, to a fascia of the patient for enhanced stability. Preferably, the shim is fixed by its eyelet to the fascia before the plug is inserted into the graft. In order to place the shim around the graft, the shim comprises a radially extending slot and is guided radially over the graft at the location of the slot. For this purpose, the slot has a width that is at least equal to or preferably exceeds the outer diameter of the graft with the catheter inserted therein. Further, the inner diameter of the shim is smaller than the outer diameter of the plug so that the plug is prevented from sliding through the shim and into the vessel.

Once the shim has been brought into the desired position around the graft and the plug has been pushed into the graft to its final position, the position of the medical device in the patient is finalized. Then, the graft is twisted so as to reduce its inner diameter. Thereby, a clamping force is exerted onto the catheter inside the graft and the slidable plug is pushed against the shim and tightly compressed on the catheter. The plug and the shim serve as a twisting stopper for the graft. Thereby, the anastomosis can be protected from being exerted to a tensile force when the graft is twisted in order to fix the catheter relative to the graft. In other words, torsional and tensile forces created by the twisting act do not reach the anastomosis and, thus, cannot negatively affect the anastomosis seam.

Once the graft has been twisted, it is held in the twisted state, e.g. by using a clamp which acts on the graft from the outside thereof, so that the graft cannot untwist again. Then the twisted graft is sutured to itself to conserve the twisted structure and, thus, to hold it constantly in the twisted state. The resulting seam may be col-linear with a longitudinal extension of the twisted graft. Then the clamp may be removed. Next, the twisted graft may be shortened to a desired length, preferably without impairing the suture of the graft.

When the graft is properly fixed to the catheter by the clamping forces generated by the twisted graft, the medical device can no longer move forwards or backwards inside the vessel. Therefore, the twisted graft is pushed into the patient's body while the catheter extends through the twisted graft out of the patient's body, and the opened skin may then be closed around the catheter.

The graft is usually made of Dacron® or a similar material. The graft is knitted, warp-knitted, knotted, woven or a nonwoven fabric so as to facilitate suturing. In order to ensure that the graft can be untwisted without efforts when the medical device is to be removed from the vessel, ingrowth of tissue into the graft should be prevented, as this would hinder untwisting. Therefore, a sleeve can be provided around the twisted graft in order to prevent any ingrowth of tissue into the graft. The sleeve may be tubular or may be wound around the twisted portion of the graft. The sleeve may be made of polytetrafluoroethylene (PTFE), such as Teflon®.

An advantage of this method is that the medical device can be removed easily without opening up the patient. More specifically, when the medical device has to be removed, the suturing on the graft that holds the graft in the twisted state is removed first. The graft is then untwisted and loses its clamping force on the catheter. The medical device is pulled out of the vessel and the graft, whereby the slidable plug is also pulled out of the graft. This is because the typically wider part (referring to the outer diameter) of the medical device arranged at the distal end of the catheter, such as the pumping device of a catheter-based intravascular blood pump, pushes the slidable plug out of the graft. Since the inner diameter of the shim exceeds the outer diameter of the medical device, removing the medical device is possible without removing the shim from the graft first. The shim may be left inside the patient.

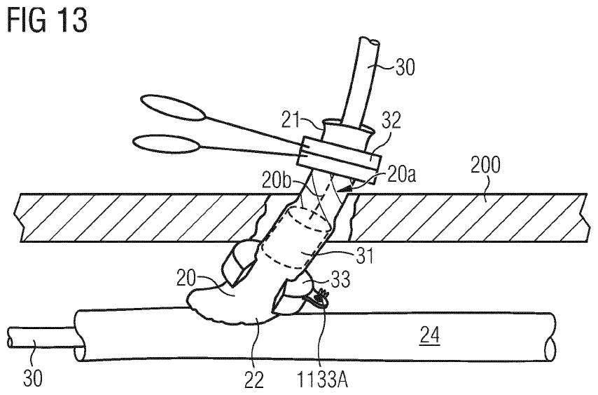

FIG. 13 shows the vascular graft 20 on the catheter 30 along with the slotted shim 33 arranged on the graft's distal end 22, the clamp 32 by which the surgeon holds the graft's proximal end 21 against the catheter 30 in order to prevent untwisting of the graft 20, and the seam 20b extending along the length of the graft's twisted portion 20a and conserving the twisted structure of the graft 20, while the proximal end of the catheter extends through the skin 200 out of the patient's body. It is also possible to twist the graft 20 and fix the twisted structure without the clamp 32 by first manually twisting the graft 20 and then providing a ligature which prevents untwisting. The distance between the patient's skin 200 and the vessel 24 may vary. It is preferred that the plug 31 is located completely below the skin 200.

The graft 20 may have an inner diameter of 6 to 10 mm, preferably 8 mm, or 8 to 10 mm. Inside the graft 20, proximal of the shim 33, the plug 31 (shown in dashed lines) is provided. With the plug 31 inserted in the graft 20, the graft 20 is sealed against the catheter to provide haemostasis. The plug 31 is particularly made of silicone rubber and has a strength of 40 to 60 shore A, preferably 50 shore A. The outer diameter of the plug 31 is smaller than the inner diameter of the graft 20. Alternatively, the graft 20 may also be elastic so that the plug 31 may have an outer diameter that equals or exceeds the inner diameter of the graft 20. The plug 31 and the shim 33 prevent the anastomosis from tensile and torsional forces incurred by the twisting movement of the graft. The graft 20 is knitted, warp-knitted, knotted, woven or a nonwoven fabric. The situation shown in FIG. 13 corresponds to the moment in the procedure before removing the clamp 32, cutting away the proximal end 21 of the graft 20, pushing the graft 20 into the patient's body and closing the opening in the patient's skin 200.

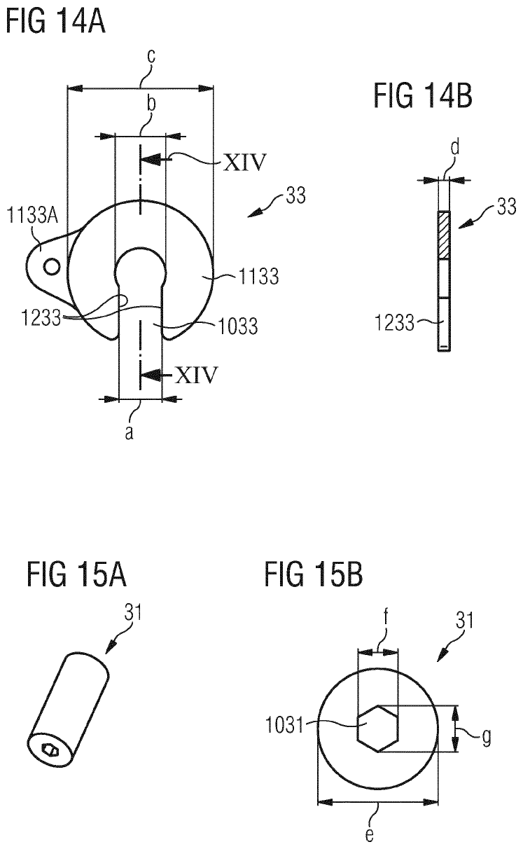

FIG. 14A shows a plan view of a slotted shim 33, such as can be used for the vascular graft twisted on the catheter as shown in FIG. 13. The slotted shim 33 has a flat main body 1133 in the form of an open ring. A flat radial protrusion 1133A extends in a radially outer direction from the open ring and forms an eyelet for fixing the shim 33 to a fascia. Spaced apart from the eyelet in a circumferential direction of the open ring, the shim 33 further comprises a slot 1033 which extends in a radial direction of the ring 33. The two ends of the open ring form respective end surfaces 1233 which define the slot 1033 and run in parallel to each other. The distance a between the end surfaces 1233, i. e. the width of the slot 1033, is between 4 and 8 mm, preferably 6 mm. The inner diameter b of the open ring is between 5 and 9 mm, preferably 7 mm. The outer diameter c of the open ring is between 15 and 25 mm, preferably 20 mm. As can be seen in FIG. 14B, which shows a sectional view of the slotted shim 33 of FIG. 14A along line XIV, the flat main body 1133 has a thickness d of 1 to 2 mm, preferably 1.5 mm. Generally, the width of the slot 1033 is dimensioned such that the graft in which the catheter is inserted fits through the slot 1033. This means that the width of the slot 1033 corresponds at least to the outer diameter of the catheter plus two times the wall thickness of the graft. Further, the inner diameter b is dimensioned such that the medical device, e. g. the pump head, which is broader than the catheter, can be guided through the shim 33. This means that the inner diameter b corresponds at least to the outer diameter of the medical device plus two times the wall thickness of the graft.

FIG. 15A shows a three-dimensional view of a plug 31, such as can be used for the vascular graft twisted on the catheter as shown in FIG. 13. The outer form of the plug 31 corresponds to a circular cylinder. The plug 31 has a length of 15 mm to 25 mm, preferably 20 mm. As can be seen in FIG. 15B, which shows a cross sectional view of the plug of FIG. 15A, the plug 31 comprises a central through hole 1031 in the form of a hexagonal cylinder. The outer diameter e of the plug 31 is 7 to 11 mm, preferably 9 mm. Respective opposing surfaces of the hexagonally shaped through hole 1031 are provided at a distance f of 2.5 mm to 3.5 mm, preferably 3 mm, while respective opposing edges are provided at a distance g of preferably 3.46 mm. Adjacent surfaces are angled at 120°. The plug 31 is made from silicone rubber and has a strength of 50 Shore A.

### Preferred Embodiments of the Disclosure

1. A surgical guide tube 1 configured for insertion into a vascular graft 20, the surgical guide tube 1 comprising:
a body 2 having a proximal end 4, a distal end 6 and a guide lumen with a longitudinal axis 8, the guide lumen extending from the proximal end 4 to the distal end 6 of the body 2 and configured for inserting a medical device, in particular a catheter 30 of an intravascular blood pump, through the body 2, and
a sideways extension 10 extending sideways from the body 2 and defining a planar surface 12a, 12b configured for being attached to a fascia 26 of a patient between a vessel 24 of the patient, preferably a blood vessel and further preferably at least one of the arteria axillaris and the arteria subclavia, and the skin of the patient,
wherein the planar surface 12a, 12b of the sideways extension 10 is inclined relative to the longitudinal axis 8 of the guide lumen by an inclination angle of about 30° to 60°, preferably 45°.

2. The surgical guide tube 1 of paragraph 1, wherein the sideways extension 10 is arranged such that its planar surface 12a, 12b comprises an axis which extends orthogonally through the longitudinal axis 8 of the guide lumen.

3. The surgical guide tube 1 of paragraph 1 or 2, wherein the sideways extension 10 is attached to the body 2 in a non-detachable manner, preferably integrally formed with the body 2.

4. The surgical guide tube 1 of paragraph 1 or 2, further comprising a separate clamping part 34 having a proximal end 36, a distal end 38 and a clamping lumen extending from the proximal end 36 to the distal end 38 of the clamping part 34, wherein the sideways extension 10 is attached to the clamping part 34 in a non-detachable manner, preferably integrally formed with the clamping part 34, and wherein the clamping part 34 is configured for clamping onto the body 2 such that the planar surface 12a, 12b of the sideways extension 10 is inclined relative to the longitudinal axis 8 of the guide lumen by said inclination angle.

5. The surgical guide 1 tube of paragraph 4, wherein the clamping part 34 is configured for clamping onto the body 2 such that a longitudinal axis of the clamping lumen is parallel to the longitudinal axis 8 of the guide lumen.

6. The surgical guide tube 1 of paragraph 4 or 5, wherein the clamping part 34 is configured for reducing the diameter of the guide lumen when clamped onto the body 2, in particular when, where the surgical guide tube 1 is provided with two or more bumps 16 or with at least one bump 16 and at least one bulge 18, 40, the clamping part 34 is placed at a longitudinal position of the surgical guide tube 1 between the bumps 16 or between the at least one bump 16 at least one bulge 18, 40.

7. The surgical guide tube 1 of any one of paragraphs 4 to 6, wherein the clamping part 34 comprises at least two arms 42 extending substantially in opposite circumferential directions and each provided with an interlocking profile 44 and arranged such that a cross-section of the clamping lumen is narrowed when the arms 42 are moved past each other and interlocked with each other via their interlocking profiles 44.

8. The surgical guide tube 1 of paragraph 7, wherein the interlocking profiles 44 of the at least two arms 42 form a ratchet mechanism.

9. The surgical guide tube 1 of any one of paragraphs 1 to 8, wherein there are provided two of said sideways extension 10, which are preferably arranged mirror-symmetrically to each other, each forming a wing, the two wings extending in opposite radial directions relative to the longitudinal axis 8 of the guide lumen of the body 2.

10. The surgical guide tube 1 of paragraph 9, wherein a distance between the longitudinal axis 8 of the guide lumen and a radially outermost edge of each of the sideways extensions 10 is between about 5 mm to about 19 mm, preferably between about 8 mm to about 16 mm, and further preferably about 12 mm.

11. The surgical guide tube 1 of any one of paragraphs 1 to 10, wherein the sideways extension 10 comprises a protrusion, a recess or an aperture 14, more preferably two or more of said aperture 14, configured for insertion of a suture 28 or clamp 32 through the sideways extension 10.

12. The surgical guide tube 1 of paragraph 11, wherein a longitudinal axis of the aperture 14 extends in a substantially vertical direction relative to the planar surface 12a, 12b.

13. The surgical guide tube 1 of paragraph 11 or 12, wherein the aperture 14 is substantially D-shaped in a transverse cross-section.

14. The surgical guide tube 1 of any one of paragraphs 1 to 13, wherein the sideways extension 10 is arranged at a proximal end 4 of the surgical guide tube 1.

15. The surgical guide tube 1 of any one of paragraphs 1 to 13, wherein a proximal bulge 40 surrounds the body 2 and forms a proximal end of the surgical guide tube 1, the proximal bulge 40 preferably being annular, further preferably having a shape of a circular ring.

16. The surgical guide tube 1 of any one of paragraphs 1 to 15, wherein the surgical guide tube 1 is provided with one or more circumferential bumps 16 extending radially from the body 2, each of said one or more circumferential bumps 16 surrounding the body 2 and preferably being annular, further preferably having a shape of a circular ring, wherein the one or more circumferential bumps 16 are arranged in spaced-apart relationship along the longitudinal axis 8 of the guide lumen.

17. The surgical guide tube 1 of paragraph 16, wherein there are at least two of said one or more circumferential bumps 16, the at least two circumferential bumps 16 comprising at least one of the features of (a) being aligned in parallel to each other along the longitudinal axis 8 of the guide lumen and (b) having a same outer diameter.

18. The surgical guide tube 1 of paragraph 16, wherein some or all of the one or more circumferential bumps 16 are positioned distal of the sideways extension 10, wherein there are preferably at least two of said distally positioned circumferential bumps and said at least two distally positioned circumferential bumps comprise at least one of the features of (a) being aligned in parallel to each other along the longitudinal axis 8 of the guide lumen and (b) having a same outer diameter.

19. The surgical guide tube 1 of any one of paragraphs 1 to 18, wherein a distal bulge 16 surrounds the body 2 and forms a distal end of the surgical guide tube 1, the distal bulge 18 preferably being annular, further preferably having a shape of a circular ring.

20. The surgical guide tube 1 of any one of paragraphs 16 to 18, wherein a distal bulge 18 surrounds the body 2 and forms a distal end of the surgical guide tube 1, the distal bulge 18 preferably being annular, further preferably having a shape of a circular ring, and wherein all of the one or more circumferential bumps 16 which are positioned proximal of said distal bulge 18 comprise at least one of the features of (a) being aligned in parallel to each other along the longitudinal axis 8 of the guide lumen and (b) having a same outer diameter.

21. The surgical guide tube 1 of paragraph 18, wherein a distal bulge 18 surrounds the body 2 and forms a distal end of the surgical guide tube 1, the distal bulge 18 preferably being annular, further preferably having a shape of a circular ring, and wherein all of the one or more circumferential bumps 16 which are positioned between the sideways extension 10 and said distal bulge 18 comprise at least one of the features of being aligned in parallel to each other along the longitudinal axis 8 of the guide lumen and having the same outer diameter.

22. The surgical guide tube 1 of any one of paragraphs 1 to 21, wherein a circumference of the distal end of the surgical guide tube 1 lies in a plane which is inclined relative to the longitudinal axis 8 of the guide lumen by about 30° to 60°, preferably 45°.

23. The surgical guide tube 1 of paragraph 22, wherein the plane is inclined, relatively to the longitudinal axis 8 of the guide lumen, in the same direction and preferably by the same inclination angle as the planar surface 12a, 12b of the sideways extension 10.

24. The surgical guide tube 1 of any one of paragraphs 16 to 23, wherein the one or more circumferential bumps 16 are arranged concentrically about the longitudinal axis 8 of the guide lumen, and an outer diameter of each of the bumps relative to the longitudinal axis of the guide lumen is about 0,8 times to about 1,8 times, preferably about 1 time to about 1,6 times, and further preferably about 1,3 times larger than an outer diameter of the body 2.

25. The surgical guide tube 1 of any one of paragraphs 1 to 24, wherein the body 2 has a constant outer diameter relative to the longitudinal axis of the guide lumen.

26. The surgical guide tube 1 of any one of paragraphs 1 to 25, including any one of paragraphs 16 to 18, 20, 22, 23 and 25 and any one of paragraphs 4 to 8, wherein all of the at least two circumferential bumps 16 are positioned along the longitudinal axis 8 of the guide lumen at a mutual distance which is larger than a distance from the proximal end 36 to the distal end 38 of the clamping part 34.

27. The surgical guide tube 1 of any one of paragraphs 1 to 26, including any one of paragraphs 16, 17, 18, 20, 22, 23, 25 and 26 and any one of paragraphs 4 to 8, wherein at least one of (a) a distance between the proximal end of the surgical guide tube 1 and one of the one or more circumferential bumps 16 which is located closest to the proximal end of the surgical guide tube 1 and (b) a distance between the distal end of the surgical guide tube 1 and one of the one or more circumferential bumps 16 which is located closest to the distal end of the surgical guide tube 1, is larger than a distance from the proximal end 36 to the distal end 38 of the clamping part 34.

28. A kit comprising the surgical guide tube 1 according to any one of paragraphs 1 to 27 and a vascular graft 20, wherein the vascular graft 20 has a proximal end configured for inserting the distal end of the surgical guide tube 1 thereinto, a distal end 22 configured for fixing it to a vessel 24 of a patient, and a graft lumen extending from the proximal end of the graft 20 to the distal end 22 of the graft 20, the graft lumen configured for accommodating a distal section of the surgical guide tube 1.

29. The kit of paragraph 28, wherein the surgical guide tube 1 comprises the one or more circumferential bumps 16 as defined in paragraph 16 and wherein a diameter of the graft lumen is between 1 time and about 1,2 times, preferably about 1,1 times, an outer diameter of the one or more circumferential bumps 16, in particular a largest outer diameter of the one or more circumferential bumps 16.

30. The kit of paragraph 28 or 29, wherein the surgical guide tube 1 comprises the clamping part 34 as defined in any one of the paragraphs 4 to 8 and wherein the clamping part 34 is configured for fixing the graft 20 to the surgical guide tube 1 by clamping the clamping part 34 onto the graft 20 while the surgical guide tube 1 is inserted in the graft 20.

31. The kit of paragraph 30, wherein the clamping part 34 is configured for reducing the diameter of the guide lumen of the surgical guide tube 1 by clamping the clamping part 34 onto the graft 20 while the surgical guide tube 1 is inserted in the graft 20.

32. The kit of any one of paragraphs 28 to 31, further comprising at least one of (a) at least one suture 28 and (b) at least one fixing clamp 32, configured for fixing the graft 20 to an outer circumference of the body 2 with the surgical guide tube 1 inserted in the graft 20.

33. The kit of paragraph 32, wherein the at least one suture 28 or the at least one fixing clamp 32 is configured for reducing the diameter of the graft 20 by applying it to the graft 20 while the surgical guide tube 1 is inserted in the graft 20.

34. The kit of paragraph 32 or 33, wherein the fixing clamp 32 is configured for reducing the diameter of the guide lumen of the graft 20 by applying the fixing clamp 32 to the graft 20 while the surgical guide tube 1 is inserted in the graft 20.

35. The kit of any one of paragraphs 28 to 34, further comprising a medical device, in particular a catheter 30, configured for inserting it into and through the guide lumen of the surgical guide tube 1, wherein the surgical guide tube 1 is preferably slidable on an outer surface of the medical device.

36. The kit of paragraph 35, further comprising an intravascular blood pump configured for connecting it to the medical device and for inserting it through the graft 20 into the vessel 24 of the patient.

37. A system configured for fixing a medical device to a patient's body, the system comprising:
(a) a surgical guide tube 1 configured for insertion into a vascular graft 20, the surgical guide tube 1 comprising
   a body 2 having a proximal end 4, a distal end 6 and a guide lumen with a longitudinal axis 8, the guide lumen extending from the proximal end 4 to the distal end 6 of the body 2 and configured for inserting a medical device, in particular a catheter 30 of an intravascular blood pump, through the body 2, and
   a sideways extension 10 extending sideways from the body 2 and defining a planar surface 12a, 12b configured for being attached to a patient's fascia 26 between a vessel of the patient, preferably a blood vessel and further preferably at least one of the arteria axillaris and the arteria subclavia, and the patient's skin, and
(b) a fixator 46 configured for fixing it to the patient's skin and for holding the medical device to the patient's body, the fixator 46 having a central body 48 and two fixator wings 50 extending from the central body 48 in opposite directions, wherein each fixator wing 50 has a planar surface facing in a common direction.

38. The system of paragraph 37, wherein the planar surface of the sideways extension 10 is inclined relative to the longitudinal axis 8 of the guide lumen by an inclination angle of about 30° to 60°, preferably 45°.

39. The system of paragraph 37 or 38, wherein the sideways extension 10 is arranged such that its planar surface 12a, 12b comprises an axis which extends orthogonally through the longitudinal axis 8 of the guide lumen.

40. The system of any one of paragraphs 37 to 39, wherein the sideways extension 10 is attached to the body 2 in a non-detachable manner, preferably integrally formed with the body 2.

41. The system of any one of paragraphs 37 to 40, the surgical guide tube 1 further comprising a separate clamping part 34 having a proximal end 36, a distal end 38 and a clamping lumen extending from the proximal end 36 to the distal end 38 of the clamping part 34, wherein the sideways extension 10 is attached to the clamping part 34 in a non-detachable manner, preferably integrally formed with the clamping part 34, and wherein the clamping part 34 is configured for clamping onto or around the body 2, preferably such that the planar surface 12a, 12b of the sideways extension 10 is inclined relative to the longitudinal axis 8 of the guide lumen by said inclination angle.

42. The system of paragraph 41, wherein the clamping part 34 is configured for clamping onto or around the body 2 such that a longitudinal axis of the clamping lumen is parallel to the longitudinal axis 8 of the guide lumen.

43. The system of paragraph 41 or 42, wherein the clamping part 34 is configured for reducing the diameter of the guide lumen when clamped onto the body 2, in particular when, where the surgical guide tube 1 is provided with two or more bumps 16 or with at least one bump 16 and at least one bulge 18, 40, the clamping part 34 is placed at a longitudinal position of the surgical guide tube 1 between the bumps 16 or between the at least one bump 16 at least one bulge 18, 40.

44. The system of any one of paragraphs 41 to 43, wherein the clamping part 34 comprises at least two arms 42 extending substantially in opposite circumferential directions and each provided with an interlocking profile 44 and arranged such that a cross-section of the clamping lumen is narrowed when the arms 42 are moved past each other and interlocked with each other via their interlocking profiles 44.

45. The system of paragraph 44, wherein the interlocking profiles 44 of the at least two arms 42 form a ratchet mechanism.

46. The system of any one of paragraphs 37 to 45, wherein there are provided two of said sideways extensions 10, each forming a wing, the two wings extending in opposite radial directions relative to the longitudinal axis 8 of the guide lumen of the body 2.

47. The system of paragraph 46, wherein a distance between the longitudinal axis 8 of the guide lumen and a radially outermost edge of each of the sideways extensions 10 is between about 5 mm and about 19 mm, preferably between about 8 mm and about 16 mm, and further preferably about 12 mm.

48. The system of any one of paragraphs 37 to 47, wherein the sideways extension 10 comprises an aperture 14, more preferably two or more of said aperture 14, configured for insertion of a suture 28 or clamp 32 through the sideways extension 10.

49. The system of paragraph 48, wherein a longitudinal axis of the aperture 14 extends in a substantially vertical direction relative to the planar surface 12a, 12b.

50. The system of paragraph 48 or 49, wherein the aperture 14 is substantially D-shaped in a transverse cross-section.

51. The system of any one of paragraphs 37 to 50, wherein the sideways extension 10 is arranged at a proximal end of the surgical guide tube 1.

52. The system of any one of paragraphs 37 to 50, wherein a proximal bulge 40 surrounds the body 2 and forms a proximal end of the surgical guide tube 1, the proximal bulge 40 preferably being annular, further preferably having the shape of a circular ring.

53. The system of any one of paragraphs 37 to 52, wherein the surgical guide tube 1 is provided with one or more circumferential bumps 16 extending radially from the body, each of said one or more circumferential bumps 16 surrounding the body 2 and preferably being annular, further preferably having the shape of a circular ring, wherein the one or more circumferential bumps 16 are arranged in spaced-apart relationship along the longitudinal axis 8 of the guide lumen.

54. The system of paragraph 53, wherein there are at least two of said one or more circumferential bumps 16, the at least two circumferential bumps 16 comprising at least one of the features of (a) being aligned in parallel to each other along the longitudinal axis 8 of the guide lumen and (b) having a same outer diameter.

55. The system of paragraph 53, wherein some or all of the one or more circumferential bumps 16 are positioned distal of the sideways extension 10, wherein there are preferably at least two of said distally positioned circumferential bumps and said at least two distally positioned circumferential bumps comprise at least one of the features of (a) being aligned in parallel to each other along the longitudinal axis 8 of the guide lumen and (b) having a same outer diameter.

56. The system of any one of paragraphs 37 to 55, wherein a distal bulge 18 surrounds the body 2 and forms a distal end of the surgical guide tube 1, the distal bulge 18 preferably being annular, further preferably having a shape of a circular ring.

57. The system of any one of paragraphs 37 to 55, wherein a distal bulge 18 surrounds the body 2 and forms a distal end of the surgical guide tube 1, the distal bulge 18 preferably being annular, further preferably having a shape of a circular ring, and wherein all of the one or more circumferential bumps 16 which are positioned proximal of said distal bulge 18 comprise at least one of the features of (a) being aligned in parallel to each other along the longitudinal axis 8 of the guide lumen and (b) having a same outer diameter.

58. The system of paragraph 55, wherein a distal bulge 18 surrounds the body 2 and forms a distal end of the surgical guide tube 1, the distal bulge 18 preferably being annular, further preferably having a shape of a circular ring, and wherein all of the one or more circumferential bumps 16 which are positioned between the sideways extension 10 and said distal bulge 18 comprise at least one of the features of (a) being aligned in parallel to each other along the longitudinal axis 8 of the guide lumen and (b) having the same outer diameter.

59. The system of any one of paragraphs 41 to 66, wherein a circumference of the distal end of the surgical guide tube 1 lies in a plane which is inclined relative to the longitudinal axis 8 of the guide lumen by about 30° to 60°, preferably 45°.

60. The system of paragraph 59, wherein the plane is inclined, relatively to the longitudinal axis 8 of the guide lumen, in the same direction and preferably by the same inclination angle as the planar surface 12a, 12b of the sideways extension 10.

61. The system of any one of paragraphs 53 to 60, wherein the one or more circumferential bumps 16 are arranged concentrically about the longitudinal axis 8 of the guide lumen and an outer diameter of each of the bumps 16 relative to the longitudinal axis of the guide lumen is about 0,8 times to about 1,8 times, preferably 1 time to about 1,6 times, and further preferably about 1,3 times larger than an outer diameter of the body 2.

62. The system of any one of paragraphs 37 to 61, wherein the body 2 has a constant outer diameter relative to the longitudinal axis of the guide lumen.

63. The system of any one of paragraphs 53 to 62, including any one of paragraphs 41 to 45 and any one of paragraphs 53 to 55, 58, 61, wherein all of the at least two circumferential bumps 16 are positioned along the longitudinal axis 8 of the guide lumen at a mutual distance which is larger than a distance from the proximal end 36 to the distal end 38 of the clamping part 34.

64. The system of any one of paragraphs 53 to 63, including any one of paragraphs 41 to 45 and any one of paragraphs 53 to 55, 58, 61, wherein at least one of (a) a distance between the proximal end of the surgical guide tube 1 and one of the one or more circumferential bumps 16 which is located closest to the proximal end of the surgical guide tube 1 and (b) a distance between the distal end of the surgical guide tube 1 and one of the one or more circumferential bumps 16 which is located closest to the distal end of the surgical guide tube 1, is larger than a distance from the proximal end 36 to the distal end 38 of the clamping part 34.

65. The system of any one of paragraphs 37 to 64 further comprising a vascular graft 20, wherein the graft 20 has a proximal end configured for inserting the distal end of the surgical guide tube 1 thereinto, a distal end 22 configured for fixing it to a patient's vessel 24 and a graft lumen extending from the proximal end of the graft 20 to the distal end 22 of the graft 20, the graft lumen configured for accommodating a distal section of the surgical guide tube 1.

66. The system of paragraph 65, wherein the surgical guide tube 1 comprises the one or more circumferential bumps 16 as defined in paragraph 53 and wherein a diameter of the graft lumen is between 1 time and about 1,2 times, preferably about 1,1 times, an outer diameter of the one or more circumferential bumps 16, in particular a largest outer diameter of the one or more circumferential bumps 16.

67. The system of paragraph 65 or 66, wherein the surgical guide tube 1 comprises the clamping part 34 as defined in any one of the paragraphs 41 to 45 and wherein the clamping part 34 is configured for fixing the graft 20 to the surgical guide tube 1 by clamping the clamping part 34 onto the graft 20 while the surgical guide tube 1 is inserted in the graft 20.

68. The system of paragraph 67, wherein the clamping part 34 is configured for reducing the diameter of the guide lumen of the surgical guide tube 1 by clamping the clamping part 34 onto the graft 20 while the surgical guide tube 1 is inserted in the graft 20.

69. The system of any one of paragraphs 65 to 68, further comprising at least one of (a) at least one suture 28 and (b) at least one fixing clamp 32, configured for fixing the graft 20 to an outer circumference of the body 2 with the surgical guide tube 1 inserted in the graft 20.

70. The system of paragraph 69, wherein the at least one suture 28 or the at least one fixing clamp 32 is configured for reducing the diameter of the graft 20 by applying it to the graft 20 while the surgical guide tube 1 is inserted in the graft 20.

71. The system of paragraph 69 or 70, wherein the fixing clamp 32 is configured for reducing the diameter of the guide lumen of the graft 20 by applying the fixing clamp 32 to the graft 20 while the surgical guide tube 1 is inserted in the graft 20.

72. The system of any one of paragraphs 65 to 72, further comprising a medical device, in particular a catheter 30, configured for inserting it into and through the guide lumen of the surgical guide tube 1, wherein the surgical guide tube 1 is preferably slidable on an outer surface of the medical device.

73. The system of paragraph 72, further comprising an intravascular blood pump configured for connecting it to the medical device and for inserting it through the graft 20 into the vessel 24 of the patient.

74. The system of any one of paragraphs 37 to 73, wherein each of the two fixator wings 50 comprises at least one aperture, preferably two or more apertures, which is configured for inserting of a suture or clamp therethrough so as to attach the fixator wings 50 to the patient's skin.

75. The system of any one of paragraphs 37 to 74, wherein the planar surfaces of the fixator wings 50 are at least partially covered with an adhesive configured for sticking the planar surfaces to the patient's skin.

76. The system of any one of paragraphs 37 to 75, further comprising a repositioning unit 54 having a proximal end 56, a distal end 58 and a repositioning lumen extending from the proximal end 56 to the distal end 58 of the repositioning unit 54, the repositioning lumen 54 configured for insertion of the medical device through the repositioning unit 54, wherein the repositioning unit 54 comprises at least one button 60, or lever, which is accessible from outside the repositioning unit 54, preferably protruding to or extending through a circumferential outer surface of the repositioning unit 54, the button 60 or lever configured, in a first state, for holding the medical device in position relative to the repositioning lumen and, in a second state, for releasing the medical device upon actuation of the button 60 or lever so as to allow movement of the medical device relative to the repositioning lumen.

77. The system of any one of paragraphs 37 to 76, wherein the central body 48 of the fixator 46 has a proximal end 62, a distal end 64 and a fixator lumen extending from the proximal end 62 to the distal end 64 of the central body 46, the fixator lumen configured for insertion of the medical device through the central body 46.

78. The system of any one of paragraphs 37 to 77, wherein a tubular extension 66 having an extension lumen is attached to, preferably integrally formed with, the central body 46 at a distal end 64 of the central body 46, the extension lumen configured for inserting the medical device through the tubular extension 66.

79. The system of any one of paragraphs 76 to 78, wherein the repositioning unit 54 is attached to the proximal end of the central body 46.

80. The system of paragraph 79, wherein the extension lumen, the fixator lumen and the repositioning lumen are aligned coaxially with respect to each other.

81. The system of any one of paragraphs 78 to 80, including paragraph 76, further comprising the medical device which is insertable through the extension lumen, the fixator lumen and the repositioning lumen, wherein the tubular extension 66, the fixator 46 and the repositioning unit 54 are slidable on the medical device when the button 60 or lever of the repositioning unit 54 is actuated.

82. The system of any one of paragraphs 37 to 76, wherein the central body 46 forms an arched bridge 80 such that the medical device can pass between the central body 46 and the patient's skin when the fixator 46 is applied to the patient's skin.

83. The system of paragraph 82, further comprising a fixation graft 96 configured for implanting it under the patient's skin and insertion of the medical device through the fixation graft 96, wherein the fixation graft 96 preferably comprises a velour on an outer surface of the fixation graft 96, the velour configured for skin to grow thereinto.

84. The system of any one of paragraphs 37 to 76, wherein a tubular extension 66 having an extension lumen is attached to, preferably integrally formed with, the surgical guide tube 1 at the proximal end of the surgical guide tube 1, the extension lumen configured for insertion of the medical device through the tubular extension 66.

85. The system of paragraph 84, including paragraph 76, further comprising a connecting portion 74 having a connecting lumen configured for insertion of the medical device through the connecting portion 74, the connecting portion 74 connecting the tubular extension 66 to the repositioning unit 54, wherein the connecting portion 74 has a section with a reduced outer diameter, said section being positioned between the repositioning unit 54 and the tubular extension 66 so as to form a waist portion 76 between the repositioning unit 74 and the tubular extension 66.

86. The system of paragraph 85, wherein the central body 48 of the fixator 46 forms an arched bridge 80 such that the waist portion 76 can pass between the central body 48 and the patient's skin when the fixator 46 is applied to the patient's skin.

87. The system of paragraph 86, wherein the waist portion 76 has a proximal end and a distal end and wherein the waist portion 76 can move under the bridge 80 along a longitudinal direction, wherein movement of the waist portion 76 along the longitudinal direction is stopped at the distal end and at the proximal end of the waist portion 76, respectively.

88. The system of paragraph 85, wherein the extension lumen, the connecting lumen and the repositioning lumen are aligned coaxially with respect to each other.

89. A clamping unit 100 configured for fixing of a medical device, in particular a catheter 30 of an intravascular blood pump, to a patient's body, the clamping unit 100 having a tubular body 102 with a proximal end 104, a distal end 106 and a lumen extending from the proximal end 104 to the distal end 106 of the tubular body 102 and configured for insertion of the medical device through the tubular body 102, wherein the tubular body 102 comprises
a surgical guide tube portion 108 at the distal end 104 of the tubular body 102 configured for inserting it into a vascular graft 20,
a sideways extension 110 defining a planar surface 112a, 112b configured for being attached to a patient's fascia 26 between a vessel 24 of the patient, preferably a blood vessel and further preferably at least one of the arteria axillaris and the arteria subclavia, and the patient's skin, wherein the sideways extension 110 is provided proximal of the surgical guide tube portion 108,
a handling portion 114 provided proximal of the sideways extension 110,
a rotatable tube 116 configured for insertion of the medical device therethrough, wherein the rotatable tube 116 is provided inside the lumen of the tubular body 102 and operatively connected to a first lever 118, or first button, which is accessible from outside the handling portion 114, preferably protruding to or extending through an outer surface of the handling portion 114, such that actuation of the first lever 118 or first button causes rotation of the rotatable tube 116 about a longitudinal axis of the rotatable tube 116 in a non-twisting manner, and
a flexible tubular foil portion 120 configured for insertion of the medical device therethrough, wherein the foil portion 120 is provided inside the lumen of the tubular body 102 and fixed to the rotatable tube 116 at a proximal connecting portion 122 of the foil portion 120 in a non-rotatable manner, wherein the foil portion 120 is further fixed to the inner lumen of the tubular body 102 at a distal connecting portion 124 of the foil portion 120 in a non-rotatable manner, thereby causing the foil portion 120 to twist when the rotatable tube 116 is caused to rotate in a first direction and to untwist, respectively, when the rotatable tube 116 is caused to rotate in a second direction opposite to the first direction after the rotatable tube 116 has been rotated in the first direction.

90. The clamping unit 100 of paragraph 89, wherein the handling portion 114 further comprises a second button 126, or second lever, which is accessible from outside the handling portion 114, preferably protruding to or extending through a circumferential outer surface of the handling portion 114, the second button 126 or second lever configured, in a first state, for holding the medical device in position relative to the lumen of the tubular body 102 and, in a second state, for releasing the medical device upon actuation of the second button 126 or second lever so as to allow movement of the medical device relative to the inner lumen.

91. The clamping unit 100 of paragraph 89 or 90, wherein the handling portion 114 comprises a section with a reduced outer diameter so as to form a waist portion 128, wherein the first lever 118, or first button, is preferably provided at the waist portion 128 and, further preferably, does not radially extend over portions of the handling portion 114 adjacent to the waist portion 128.

92. The clamping unit 100 of any one of paragraphs 89 to 91, further comprising a fixator 46 configured for fixation to the patient's skin and for holding the clamping unit 100 against the patient's body, the fixator 46 having a central body 48 and two fixator wings 50 extending from the central body 46 in opposite directions, wherein each fixator wing 50 has a planar surface facing in a common direction, wherein the central body 48 of the fixator 46 forms an arched bridge 80 such that the waist portion 128 of the handling portion 114 of the tubular body 102 can pass between the central body 48 of the fixator 46 and the patient's skin when the fixator 46 is applied to the patient's skin.

93. The clamping unit 100 of paragraph 92, wherein the waist portion 128 has a proximal end and a distal end and wherein the waist portion 128 can move under the bridge 80 along a longitudinal direction, wherein movement of the waist portion 80 along the longitudinal direction is stopped at the distal end and at the proximal end of the waist portion 128, respectively.

94. The clamping unit 100 of any one of paragraphs 89 to 93, wherein at least one of the surgical guide tube portion 108, the sideways extension 110 and the fixator 46 has the same configuration as the surgical guide tube 1, the sideways extension 10 and the fixator 46, respectively, of the system according to any one of paragraphs 37 to 88.

95. A method of providing a vascular access to a vessel of a patient, preferably a blood vessel and further preferably at least one of the arteria axillaris and the arteria subclavia, comprising the steps of providing a surgical guide tube comprising a body having a proximal end, a distal end, and a guide lumen with a longitudinal axis, the guide lumen extending from the proximal end to the distal end and configured for insertion of a medical device, in particular a catheter of an intravascular blood pump, through the surgical guide tube and fixing the surgical guide tube to a fascia of the patient between the vessel and skin of the patient.

96. The method of paragraph 95, wherein the surgical guide tube further comprises a sideways extension extending sideways from the body and defining a planar surface, the method further comprising the step of attaching the planar surface of the sideways extension to the patient's fascia, preferably by suturing, clamping or sticking the sideways extension to the fascia.

97. The method of paragraph 96, wherein the sideways extension comprises a proximal planar surface and a distal planar surface and the sideways extension is attached to the fascia with the proximal planar surface.

98. The method of paragraph 96, wherein the sideways extension comprises a proximal planar surface and a distal planar surface and the sideways extension is attached to the fascia with the distal planar surface of the sideways extension.

99. The method of any one of paragraphs 95 to 98, wherein the surgical guide tube has the same configuration as the surgical guide tube of the system according to any one of paragraphs 37 to 64.

100. The method of any one of paragraphs 95 to 99, further comprising the step of fixing a distal end of a vascular graft to the patient's vessel, preferably by anastomosis, wherein the vascular graft has a graft lumen extending from the proximal end to the distal end of the graft, the graft lumen configured for accommodating a distal section of the surgical guide tube in a proximal end of the vascular graft.

101. The method of paragraph 100, further comprising the step of inserting an intravascular blood pump comprising a catheter at its proximal end into and through the vascular graft and further into the patient's vessel, and preferably comprising the additional step of advancing the intravascular pump to an intended final position which is preferably a left ventricle of the patient's heart, wherein the surgical guide tube is slidably mounted on an outer surface of the catheter and wherein the method further comprises the step of sliding the surgical guide tube on the outer surface of the catheter such that the distal section of the surgical guide tube is accommodated in the graft lumen at the proximal end of the vascular graft.

102. The method of paragraph 101, wherein a diameter of the graft lumen is between 1 time and about 1,2 times, preferably about 1,1 times, an outer diameter of the surgical guide tube.

103. The method of paragraph 101 or 102, further comprising the step of fixing the vascular graft to an outer surface of the surgical guide tube, after the surgical guide tube has been accommodated in the graft lumen of the vascular graft, preferably by clamping or suturing of the vascular graft to the outer surface of the surgical guide tube.

104. The method of paragraph 103, wherein the surgical guide tube further comprises a separate clamping part having a proximal end, a distal end and a clamping lumen extending from the proximal end to the distal end of the clamping part, wherein the sideways extension is attached to the clamping part in a non-detachable manner, preferably integrally formed with the clamping part, and wherein the clamping part is configured for clamping onto or around the body of the surgical guide tube, and wherein the method comprises the step of fixing the graft to the body of the surgical guide tube by clamping the clamping part onto the vascular graft in which the distal section of the surgical guide tube is accommodated.

105. The method of paragraph 104, wherein the clamping part reduces the diameter of the guide lumen when clamped onto the vascular graft in which the distal section of the surgical guide tube is accommodated, in particular, where the surgical guide tube is provided with two or more bumps or with at least one bump and at least one bulge, at a longitudinal position of the surgical guide tube between the bumps or between the at least one bump at least one bulge, thereby fixing the medical device inside the guide lumen such that movement of the medical device along the guide lumen is prevented.

106. The method of paragraph 103, further comprising the step of fixing the vascular graft to the body of the surgical guide tube, the distal section of which has been accommodated in the vascular graft, by applying at least one of at least one suture and at least one fixing clamp onto the graft in which the distal section of the surgical guide tube is accommodated, in particular, where the surgical guide tube is provided with two or more bumps or with at least one bump and at least one bulge, at a position of the surgical guide tube between the bumps or between the at least one bump and the at least one bulge.

107. The method of paragraph 106, wherein the at least one suture or the at least one fixing clamp reduces a diameter of the graft lumen when applied to the vascular graft in which the proximal section of the surgical guide tube is accommodated, in particular to a reduced diameter which is smaller than an outer diameter of the bumps or the at least one bump at least one bulge, thereby reducing, preferably preventing, movement of the surgical guide tube along the graft lumen.

108. The method of paragraph 106 or 107, wherein the fixing clamp reduces a diameter of the guide lumen when clamped onto the vascular graft in which the distal section of the surgical guide tube is inserted, in particular when the fixing clamp is placed at a position of the surgical guide tube between the bumps or between the at least one bump and the at least one bulge, thereby fixing the medical device inside the guide lumen such that movement of the medical device along the guide lumen is prevented.

109. The method of any one of paragraphs 95 to 108, further comprising the step of fixing a fixator to the patient's skin such that the medical device is held to the patient's body, wherein the fixator has a central body and two fixator wings extending from the central body in opposite directions, wherein each fixator wing has a planar surface facing in a common direction.

110. The method of paragraph 109, wherein each of the two fixator wings comprises at least one aperture, preferably two or more apertures, and wherein the method comprises the step of inserting a suture or clamp through said at least one aperture such that the fixator wings are attached to the patient's skin.

111. The method of paragraph 109 or 110, wherein the planar surfaces of the fixator wings are at least partially covered with an adhesive and wherein the method further comprises the step of sticking the planar surfaces to the patient's skin.

112. The method of any one of paragraphs 109 to 111, wherein the central body of the fixator has a proximal end, a distal end and a fixator lumen extending from the proximal end to the distal end of the central body, wherein the method further comprises the step of inserting the medical device into and through the fixator lumen, wherein the central body of the fixator is preferably slidably mounted on the medical device.

113. The method of any one of paragraphs 109 to 112, wherein a tubular extension having an extension lumen is attached to, preferably integrally formed with, the central body of the fixator at the distal end of the central body, wherein the method further comprises the step of inserting the medical device into and through the extension lumen, wherein the tubular extension is preferably slidably mounted on the catheter.

114. The method of any one of paragraphs 109 to 113, wherein the method comprises the step of providing a repositioning unit having a proximal end, a distal end and a repositioning lumen extending from the proximal end to the distal end of the repositioning unit, wherein the method further comprises the step of inserting the medical device into and through the repositioning lumen, wherein the repositioning unit comprises at least one button or lever which is accessible from outside the repositioning unit, preferably protruding to or extending through a circumferential outer surface of the repositioning unit, the button or lever, in a first state, holding the medical device in its longitudinal position relative to the repositioning lumen and, in a second state, releasing the medical device upon actuation of the button or lever so as to allow movement of the medical device relative to the repositioning lumen, wherein the repositioning unit is preferably attached to the proximal end of the central body of the fixator.

115. The method of any one of paragraphs 109 to 114, further comprising the step of attaching the fixator to a location of the patient's skin such that a section of the medical device which extends from the fixator to the surgical guide tube is placed in a C-curve on the patient's skin.

116. The method of any one of paragraphs 109 to 115, wherein the central body of the fixator forms an arched bridge and wherein the method comprises the step of applying the fixator over the medical device onto the patient's skin such that the medical device passes between the central body of the fixator and the patient's skin.

117. The method of any one of paragraphs 109 to 116, comprising the step of inserting the medical device into and through a fixation graft, which preferably comprises a velour on an outer surface of the fixation graft, the velour configured for skin to grow thereinto, and the method further comprising the step of implanting the fixation graft under the patient's skin such that a portion of the medical device between the surgical guide tube and the fixator extends under the skin.

118. The method of any one of paragraphs 95 to 111, wherein a tubular extension having an extension lumen is attached to, preferably integrally formed with, the surgical guide tube at the proximal end of the surgical guide tube and wherein the method further comprises the step of inserting the medical device into and through the extension lumen.

119. The method of paragraph 118, including paragraph 114, wherein a connecting portion having a connecting lumen connects the tubular extension to a repositioning unit, wherein the method comprises the step of inserting the medical device into and through the connecting lumen, and wherein the connecting portion has a section with a reduced outer diameter, said section of reduced diameter being positioned between the repositioning unit and the tubular extension so as to form a waist portion between the repositioning unit and the tubular extension.

120. The method of paragraph 119, wherein the central body of the fixator forms an arched bridge and wherein the method comprises the step of applying the arched bridge of the fixator over the waist portion of the connecting portion onto the patient's skin such that the waist portion passes between the central body of the fixator and the patient's skin.

121. The method of paragraph 120, wherein the waist portion of the connecting portion has a proximal end and a distal end and the waist portion can move under the arched bridge of the fixator along a longitudinal direction, wherein movement of the waist portion along the longitudinal direction is stopped at the distal end and at the proximal end of the waist portion, respectively.

122. The method of any one of paragraphs 95 to 112, wherein the surgical guide tube and the sideways extension form part of a clamping unit according to any one of paragraphs 89 to 91.

123. The method of paragraph 122, including paragraph 109, wherein the central body of the fixator forms an arched bridge and wherein the method comprises the step of applying the fixator over the waist portion of the clamping unit onto the patient's skin such that the waist portion passes between the central body of the fixator and the patient's skin.

124. The method of paragraph 123, wherein the waist portion of the clamping unit has a proximal end and a distal end and can move under the arched bridge of the fixator along a longitudinal direction, wherein movement of the waist portion along the longitudinal direction is stopped at the distal end and at the proximal end of the waist portion, respectively.

125. A method of stabilizing a medical device, which comprises a catheter, in a vessel of a patient, comprising the steps:
opening the patient's skin to get access to the vessel,
connecting a vascular graft to the vessel by anastomosis,
introducing the medical device through the graft into the vessel so that the catheter extends through the graft, and
twisting the graft so as to reduce its inner diameter, thereby clamping the catheter inside the graft.

126. The method of paragraph 125, further comprising the step of holding the twisted graft in a twisted state so that it cannot untwist.

127. The method of paragraph 126, comprising the step of clamping a proximal part of the twisted graft against the catheter using a clamp.

128. The method of paragraph 126 or 127, comprising the step of suturing the twisted graft to hold it in the twisted state, preferably after the proximal part of the twisted graft has been clamped against the catheter.

129. The method of paragraph 128, further comprising the step of shortening the twisted graft prior to or after suturing it.

130. The method of paragraph 125, wherein the step of connecting the graft to the vessel by anastomosis includes suturing the graft to the vessel to create a seam between the graft and the vessel.

131. The method of paragraph 130, further comprising the step of enforcing the seam with glue.

132. The method of paragraph 125, wherein the medical device is an intravascular blood pump and the step of introducing the medical device into the vessel includes advancing the intravascular blood pump into the patient's arteria axillaris or arteria subclavia until a pumping device of the intravascular blood pump reaches into the patient's heart.

133. The method of paragraph 125, wherein the medical device comprises a plug which is slidable along the catheter and the step of introducing the medical device into the vessel comprises arranging the slidable plug on the catheter inside the graft so that the slidable plug abuts the vessel or is in close proximity thereto.

134. The method of paragraph 133, wherein the slidable plug has a closed form, preferably a closed cylindrical form, wherein particularly preferably the outer form of the slidable plug corresponds to a circular cylinder.

135. The method of paragraph 134, wherein the slidable plug has a through hole through which the catheter can extend, said through hole having a hexagonal shape in cross section and preferably the form of a hexagonal cylinder.

136. The method of paragraph 133, wherein the slidable plug is made of silicone rubber.

137. The method of paragraph 133, wherein the slidable plug has a strength of 50 shore A.

138. The method of paragraph 133, further comprising the step of placing a slotted shim around the graft, preferably prior to arranging the plug inside the graft, at a location distal of the intended position of the slidable plug inside the graft, wherein the shim preferably comprises an eyelet and the method further comprises the step of fixing the eyelet to a fascia of the patient.

139. The method of paragraph 138, wherein the step of twisting the graft so as to reduce its inner diameter includes holding the slotted shim, thereby creating a counter force against torsion forces caused by the twisting step.

140. The method of paragraph 138, wherein the slotted shim is not elastic and is preferably made of PEEK.

141. The method of paragraph 125, further comprising the steps of pushing the twisted graft into the patient's body while the catheter extends through the twisted graft and closing the opened skin around the catheter.

142. The method of paragraph 125, wherein a sleeve is provided around the twisted graft, the sleeve preventing ingrowth of tissue into the graft.

143. A kit for stabilizing a medical device which comprises a catheter 30, the kit comprising
a vascular graft 20 through which the medical device can be introduced and through which the catheter 30 can extend and
a plug 31 which is configured to be slidable along the catheter 30 and which is insertable into the graft 20, preferably while being placed on the catheter 30.

144. The kit of claim 143, wherein the graft 20 is knitted, warp-knitted, knotted, woven or a nonwoven fabric.

145. The kit of claim 143 or 144, wherein the graft 20 is twistable so as to reduce its inner diameter, thereby being configured to clamp the catheter 30 inside the graft 20.

146. The kit of any one of claims 143 to 145, wherein the graft 20 is suturable, preferably suturable in a twisted state.

147. The kit of any one of claims 143 to 146, wherein the plug 31 has a closed form, preferably a closed cylindrical form, wherein particularly preferably the outer form of the slidable plug 31 corresponds to a circular cylinder.

148. The kit of any one of claims 143 to 146, wherein the plug 31 has a through hole through which the catheter 30 can extend.

149. The kit of claim 148, wherein the through hole has a hexagonal shape in cross section and preferably in the form of a hexagonal cylinder.

150. The kit of any one of claims 143 to 149, wherein the plug 31 is made of silicone rubber.

151. The kit of any one of claims 143 to 150, wherein the plug 31 has a strength of 50 shore A.

152. The kit of any one of claims 143 to 151, further comprising a slotted shim 33 configured for being placed around the graft 20.

153. The kit of claim 152, wherein the slotted shim 33 is not elastic and preferably made of PEEK.

154. The kit of claim 152 or 153, wherein the slotted shim 33 comprises a radially extending slot through which the graft can be passed while the catheter extends through the graft.

155. The kit of any one of claims 152 to 154, wherein the shim 33 is provided with an eyelet 1133A configured for being fixed to a patient's fascia.

156. The kit of any one of claims 152 to 155, wherein an inner diameter of the slotted shim 33 is smaller than an outer diameter of the plug 31.

156. The kit of any one of claims 152 to 155, wherein an inner diameter of the slotted shim 33 is larger than an outer diameter of the medical device.

157. The kit of any one of claims 143 to 156, further comprising an intravascular blood pump as the medical device.

158. The kit of any one of claims 145 to 157, further comprising a clamp 32 configured for clamping a proximal part of the twisted graft 20 against the catheter 30.

### LIST OF REFERENCE SIGNS

1 surgical guide tube
2 tubular body
4 proximal end of the tubular body
6 distal end of the tubular body
8 longitudinal axis of the guide lumen
10 sideways extension
12a proximal planar surface
12b distal planar surface
14 aperture
16 circumferential bump
18 distal bulge
20 vascular graft/graft
20a twisted portion of the graft
20b suture on twisted portion of the graft
21 proximal end of the graft
22 distal end of the graft
24 vessel
26 fascia
28 sutures for fixing surgical guide tube to graft
29 sutures for fixing sideways extension to fascia
30 catheter
31 plug
32 fixing clamp
33 slotted shim
34 clamping part
36 proximal end of the separate clamping part
38 distal end of the proximal clamping part
40 proximal bulge
42 arm of clamping part
44 interlocking profiles
46 fixator
48 central body of the fixator
50 fixator wings
52 aperture of the fixator wing
54 repositioning unit
56 proximal end of the repositioning unit
58 distal end of the repositioning unit
60 button of the repositioning unit
62 proximal end of the central body
64 distal end of the central body
66 tubular extension
68 sleeve portion
70 distal portion of the tubular extension
72 tapering portion
74 connecting portion
76 waist portion
78 central region
80 bridge
82 guide sleeve
84 sealing
86 proximal sleeve
88 distal portion of the connecting portion
90 engagement structure of the connecting portion
92 engagement structure of the tubular extension
94 guide tube
96 fixation graft
98 location
100 clamping unit
102 tubular body
104 proximal end of the tubular body
106 distal end of the tubular body
108 surgical guide tube portion
110 sideways extension
112a proximal planar surface
112b distal planar surface
114 handling portion
116 rotatable tube
118 lever
120 tubular foil portion
122 proximal connecting portion
124 distal connecting portion
126 button
128 waist portion
130 central region
200 skin
1031 through hole
1033 slot
1133 main body
1133A eyelet
1233 end surfaces
a distance
b inner diameter
c outer diameter
d span of longitudinal movement
t thickness
e outer diameter
f distance
g distance
A virtual rotational axis

1. A surgical guide tube configured for insertion into a vascular graft, the surgical guide tube comprising:
a body having a proximal end, a distal end and a guide lumen with a longitudinal axis, the guide lumen extending from the proximal end to the distal end of the body and configured for inserting a medical device, in particular a catheter of an intravascular blood pump, through the body, and
a sideways extension extending sideways from the body and defining a planar surface configured for being attached to a fascia of a patient between a vessel of the patient, preferably a blood vessel and further preferably at least one of arteria axillaris and arteria subclavia, and a skin of the patient,
wherein the planar surface of the sideways extension is inclined relative to the longitudinal axis of the guide lumen by an inclination angle of about 30° to 60°.

2. The surgical guide tube of claim 1, further comprising a separate clamping part having a proximal end, a distal end and a clamping lumen extending from the proximal end to the distal end of the clamping part, wherein the sideways extension is attached to the clamping part in a non-detachable manner, preferably integrally formed with the clamping part, and wherein the clamping part is configured for clamping onto the body such that the planar surface of the sideways extension is inclined relative to the longitudinal axis of the guide lumen by said inclination angle.

3. The surgical guide tube of claim 2, wherein the clamping part comprises at least two arms extending substantially in opposite circumferential directions and each provided with an interlocking profile and arranged such that a cross-section of the clamping lumen is narrowed when the arms are moved past each other and interlocked with each other via their interlocking profiles.

4. The surgical guide tube of claim 1, wherein a circumference of a distal end of the surgical guide tube (1) lies in a plane which is inclined relative to the longitudinal axis (8) of the guide lumen by about 30° to 60°.

5. A kit comprising a surgical guide tube comprising—a body having a proximal end, a distal end and a guide lumen with a longitudinal axis, the guide lumen extending from the proximal end to the distal end of the body and configured for inserting a medical device, in particular a catheter of an intravascular blood pump, through the body, and
a sideways extension extending sideways from the body and defining a planar surface configured for being attached to a fascia of a patient between a vessel of the patient, preferably a blood vessel and further preferably at least one of arteria axillaris and arteria subclavia, and a skin of the patient,
wherein the planar surface of the sideways extension is inclined relative to the longitudinal axis of the guide lumen by an inclination angle of about 30° to 60°,
and a vascular graft, wherein the vascular graft has a proximal end configured for inserting the distal end of the surgical guide tube thereinto, a distal end configured for fixing it to a vessel of a patient and a graft lumen extending from the proximal end of the graft to the distal end of the graft, the graft lumen configured for accommodating a distal section of the surgical guide tube.

6. The kit of claim 5, wherein the surgical guide tube comprises a-clamping part having a proximal end, a distal end and a clamping lumen extending from the proximal end to the distal end of the clamping part, wherein the sideways extension is attached to the clamping part in a non-detachable manner, preferably integrally formed with the clamping part, and wherein the clamping part is configured for clamping onto the body such that the planar surface of the sideways extension is inclined relative to the longitudinal axis of the guide lumen by said inclination angle and wherein the clamping part is configured for fixing the graft to the surgical guide tube by clamping the clamping part onto the graft while the surgical guide tube is inserted in the graft.

7. The kit of claim 5, further comprising a medical device, in particular a catheter, configured for inserting it into and through the guide lumen of the surgical guide tube, wherein the surgical guide tube is preferably slidable on an outer surface of the medical device.

8. A system configured for fixing a medical device to a patient's body, the system comprising:
(a) a surgical guide tube configured for insertion into a vascular graft, the surgical guide tube comprising
a body having a proximal end, a distal end and a guide lumen with a longitudinal axis, the guide lumen extending from the proximal end to the distal end of the body and configured for inserting a medical device, in particular a catheter of an intravascular blood pump, therethrough, and
a sideways extension extending sideways from the body and defining a planar surface configured for being attached to a patient's fascia between a vessel of the patient, preferably a blood vessel and further preferably at least one of arteria axillaris and arteria subclavia, and a skin of the patient and
(b) a fixator configured for fixing it to the patient's skin and for holding the medical device to the patient's body, the fixator having a central body and two fixator wings extending from the central body in opposite directions, wherein each fixator wing has a planar surface facing in a common direction.

9. The system of claim 8, further comprising a repositioning unit having a proximal end, a distal end and a repositioning lumen extending from the proximal end to the distal end of the repositioning unit, the repositioning lumen configured for insertion of the medical device through the repositioning unit, wherein the repositioning unit comprises at least one button, or lever, which is accessible from outside the repositioning unit, preferably protruding to or extending through a circumferential outer surface of the repositioning unit, the button or lever configured for, in a first state, holding the medical device in its longitudinal position relative to the repositioning lumen and, in a second state, for releasing the medical device upon actuation of the button or lever so as to allow movement of the medical device relative to the repositioning lumen.

10. The system of claim 8, wherein the central body forms an arched bridge such that the medical device can pass between the central body and the patient's skin when the fixator is applied to the patient's skin.

11. The system of claim 10, further comprising a fixation graft configured for implanting it under the patient's skin and insertion of the medical device through the fixation graft, wherein the fixation graft preferably comprises a velour on an outer surface of the fixation graft, the velour configured for tissue under the skin to grow thereinto.

12. The system of claim 8, wherein a tubular extension having an extension lumen is attached to, preferably integrally formed with, the surgical guide tube at the proximal end of the surgical guide tube, the extension lumen configured for insertion of the medical device through the tubular extension.

13. The system of claim 12, further comprising a connecting portion having a connecting lumen configured for insertion of the medical device through the connecting portion, the connecting portion connecting the tubular extension to the repositioning unit, wherein the connecting portion has a section with a reduced outer diameter, said section being positioned between the repositioning unit and the tubular extension so as to form a waist portion between the repositioning unit and the tubular extension.

14. The system of claim 13, wherein the central body of the fixator forms an arched bridge such that the waist portion can pass between the central body and the patient's skin when the fixator is applied to the patient's skin.

15. A clamping unit configured for fixing of a medical device, in particular a catheter of an intravascular blood pump, to a patient's body, the clamping unit having a tubular body with a proximal end, a distal end and a lumen extending from the proximal end to the distal end of the tubular body and configured for insertion of the medical device through the tubular body, wherein the tubular body comprises
a surgical guide tube portion at the distal end (106) of the tubular body configured for inserting it into a vascular graft,
a sideways extension defining a planar surface configured for being attached to a patient's fascia between a vessel of the patient, preferably a blood vessel and further preferably at least one of arteria axillaris and arteria subclavia, and the patient's skin, wherein the sideways extension is provided proximal of the surgical guide tube portion,
a handling portion provided proximal of the sideways extension,
a rotatable tube configured for insertion of the medical device therethrough, wherein the rotatable tube is provided inside the lumen of the tubular body and operatively connected to a first lever or first button, which is accessible from outside the handling portion, preferably protruding to or extending through an outer surface of the handling portion, such that actuation of the first lever or first button causes rotation of the rotatable tube about a longitudinal axis of the rotatable tube in a non-twisting manner, and
a flexible tubular foil portion configured for insertion of the medical device therethrough, wherein the foil portion is provided inside the lumen of the tubular body and fixed to the rotatable tube at a proximal connecting portion of the foil portion in a non-rotatable manner, wherein the foil portion is further fixed to the lumen of the tubular body at a distal connecting portion of the foil portion in a non-rotatable manner, thereby causing the foil portion to twist when the rotatable tube is caused to rotate in a first direction and to untwist, respectively, when the rotatable tube is caused to rotate in a second direction opposite to the first direction after the rotatable tube has been rotated in the first direction.
